SEQUENCE LISTING (44 sequences)

SEQ ID NO: 1 - DNA, 63 bp, Artificial Sequence
  Synthetic oligonucleotide
taacacattgatgcagaaaaacagcataccatgagaggcagagtgtggaagtcagagaaaccc

SEQ ID NO: 2 - DNA, 80 bp, Artificial Sequence
  Synthetic oligonucleotide
tctcctcctccaggagggtttctctgacttccacactctgcctctcatggtatgctgtttttctgcatcaatgtgttaac

SEQ ID NO: 3 - DNA, 60 bp, Artificial Sequence
  Synthetic oligonucleotide
cgtcaaggcaaacatgccatctccttctcctgattattttacatggaatctcacctggat

SEQ ID NO: 4 - DNA, 79 bp, Artificial Sequence
  Synthetic oligonucleotide
gggaaggagtctttcattatccaggtgagattccatgtaaaataatcaggagaaggagatggcatgtttgccttgacgc

SEQ ID NO: 5 - DNA, 64 bp, Artificial Sequence
  Synthetic oligonucleotide
cgtctgagatagtaaggatgtgcggcgtgtgcctggagtagccccgactcttgtacggtcggca

SEQ ID NO: 6 - DNA, 82 bp, Artificial Sequence
  Synthetic oligonucleotide
ttctcactggtctcagatgccgaccgtacaagagtcggggctactccaggcacacgccgcacatccttactatctcagacgg

SEQ ID NO: 7 - DNA, 59 bp, Artificial Sequence
  Synthetic oligonucleotide
tactgcagggctcctttgcgcccaactcacagagaagcggctacggggcgggcgccggc

SEQ ID NO: 8 - DNA, 74 bp, Artificial Sequence
  Synthetic oligonucleotide
cgaggcgaaggcgccggcgcccgccccgtagccgcttctctgtgagttgggcgcaaaggagccctgcagtaatc

SEQ ID NO: 9 - DNA, 50 bp, Artificial Sequence
  Synthetic oligonucleotide
agtggcacatcaaacacccgtgctcacccttccccttcctcgtctacatg

SEQ ID NO: 10 - DNA, 68 bp, Artificial Sequence
  Synthetic oligonucleotide
gtactaacccatgggccatgtagacgaggaaggggaagggtgagcacgggtgtttgatgtgccactca

SEQ ID NO: 11 - DNA, 58 bp, Artificial Sequence
  Synthetic oligonucleotide
atgatggtgaagtagttcaagctcgaccccagccaagtccgattccgaagccctgagg

SEQ ID NO: 12 - DNA, 75 bp, Artificial Sequence
  Synthetic oligonucleotide
ggattcggtctggccctcagggcttcggaatcggacttggctggggtcgagcttgaactacttcaccatcattac

SEQ ID NO: 13 - DNA, 60 bp, Artificial Sequence
  Synthetic oligonucleotide
tagtgcgagttggaatgtgtctgaagctatctatgaagagcaagatgggaaggagattat

SEQ ID NO: 14 - DNA, 79 bp, Artificial Sequence
  Synthetic oligonucleotide
acatgagagggctctaaataatctccttcccatcttgctcttcatagatagcttcagacacattccaactcgcactact

SEQ ID NO: 15 - DNA, 60 bp, Artificial Sequence
  Synthetic oligonucleotide
cgacaggactcatctccttcttaactcatgagcctaaagcatctgattctaggctcatct

SEQ ID NO: 16 - DNA, 76 bp, Artificial Sequence
  Synthetic oligonucleotide
agtgttcagactgggaagatgagcctagaatcagatgctttaggctcatgagttaagaaggagatgagtcctgtcg

SEQ ID NO: 17 - DNA, 59 bp, Artificial Sequence
  Synthetic oligonucleotide
ggactgtcagttaacctgagacgtctcggttccaggctctgcactcttagtacaaccca

SEQ ID NO: 18 - DNA, 81 bp, Artificial Sequence
  Synthetic oligonucleotide
ctcacatacagaccacttaatgggttgtactaagagtgcagagcctggaaccgagacgtctcaggttaactgacagtcctc

SEQ ID NO: 19 - DNA, 66 bp, Artificial Sequence
  Synthetic oligonucleotide
gcctctatctgaaaactcagaccgatttggccatagattattagctctgagaaacagtgtgtctga

SEQ ID NO: 20 - DNA, 84 bp, Artificial Sequence
  Synthetic oligonucleotide
cactacacacacactctctcagacacactgtttctcagagctaataatctatggccaaatcggtctgagttttcagatagaggc

SEQ ID NO: 21 - DNA, 65 bp, Artificial Sequence
  Synthetic oligonucleotide
gcctagcgtctttgtgaacgtataaagctgggtgcttttaggagcacccaagtcacctcttgaat

SEQ ID NO: 22 - DNA, 79 bp, Artificial Sequence
  Synthetic oligonucleotide
ccaagcagcaaagcattcaagaggtgacttgggtgctcctaaaagcacccagctttatacgttcacaaagacgctaggc

SEQ ID NO: 23 - DNA, 63 bp, Artificial Sequence
  Synthetic oligonucleotide
gtacagtgcactgaccatttaatacacatggggtaacctttggggcatcctgccattatgtct

SEQ ID NO: 24 - DNA, 77 bp, Artificial Sequence
  Synthetic oligonucleotide
ccagaggctgtgcagacataatggcaggatgccccaaaggttaccccatgtgtattaaatggtcagtgcactgtacg

SEQ ID NO: 25 - DNA, 67 bp, Artificial Sequence
  Synthetic oligonucleotide
acaagcatgtaatcttgctttcctacaccactaccttttcatgtatcctggcttcgtttccatgttg

SEQ ID NO: 26 - DNA, 89 bp, Artificial Sequence
  Synthetic oligonucleotide
ttaggtcatttataggcctccaacatggaaacgaagccaggatacatgaaaaggtagtggtgtaggaaagcaagattacatgcttgtct

SEQ ID NO: 27 - DNA, 58 bp, Artificial Sequence
  Synthetic oligonucleotide
cccactcaagtatgaaagcacgggaacgtgagttcagaagagagagatatcaaagagg

SEQ ID NO: 28 - DNA, 82 bp, Artificial Sequence
  Synthetic oligonucleotide
attccaaatgcttaatggatatttcctctttgatatctctctcttctgaactcacgttcccgtgctttcatacttgagtggg

SEQ ID NO: 29 - DNA, 70 bp, Artificial Sequence
  Synthetic oligonucleotide
gaggaagcgatagtgagaatgagcagctgcaggagcactgcgccatggccatttaccaggtgcagtgaac

SEQ ID NO: 30 - DNA, 84 bp, Artificial Sequence
  Synthetic oligonucleotide
aaccgctgggagagttcactgcacctggtaaatggccatggcgcagtgctcctgcagctgctcattctcactatcgcttcctcc

SEQ ID NO: 31 - DNA, 65 bp, Artificial Sequence
  Synthetic oligonucleotide
cttgaacaggtgtccaaagccagaagggcctaaagcagcactgccacccccactgccacttgctt

SEQ ID NO: 32 - DNA, 84 bp, Artificial Sequence
  Synthetic oligonucleotide
caacctaagaagtccaagaaagcaagtggcagtgggggtggcagtgctgctttaggcccttctggctttggacacctgttcaag

SEQ ID NO: 33 - DNA, 55 bp, Artificial Sequence
  Synthetic oligonucleotide
atgtccgaagtagctattttatcacatagtcattcttctaatacccctctgctca

SEQ ID NO: 34 - DNA, 78 bp, Artificial Sequence
  Synthetic oligonucleotide
agcatagggaagaagaattagtgagcagaggggtattagaagaatgactatgtgataaaatagctacttcggacatca

SEQ ID NO: 35 - DNA, 76 bp, Artificial Sequence
  Synthetic oligonucleotide
ttcctgcagggaaacagcatcgattgttttctttaaaagatcccctactcctttttggctaactgaaccctgactt

SEQ ID NO: 36 - DNA, 59 bp, Artificial Sequence
  Synthetic oligonucleotide
gtcagggttcagttagccaaaaaggagtaggggatcttttaaagaaaacaatcgatgct

SEQ ID NO: 37 - DNA, 29 bp, Artificial Sequence
  Synthetic oligonucleotide
ggtaaagaaactaaagcaatcagaaagga

SEQ ID NO: 38 - DNA, 82 bp, Artificial Sequence
  Synthetic oligonucleotide
gggtcactgccatggaggagccgcagtcagatcctagcgtcgagccccctctgagtcaggaaacattttcagacccgcaacg

SEQ ID NO: 39 - DNA, 68 bp, Artificial Sequence
  Synthetic oligonucleotide
cgttgcgggtctgaaaatgtttcctgactcagagggggctcgacgctaggatctgactgcggctcctc

SEQ ID NO: 40 - DNA, 25 bp, Artificial Sequence
  Synthetic oligonucleotide
aatggatccactcacagtttccata

SEQ ID NO: 41 - DNA, 136 bp, Artificial Sequence
  Synthetic oligonucleotide
gccactagcaccatttacagccagagcctctgcttcgggagatggtctctcttgggggcgctttcctgcagggaaacagcatcgattgttttctttaaaagatcccctactcctttttggctaactgaaccctgac

SEQ ID NO: 42 - DNA, 121 bp, Artificial Sequence
  Synthetic oligonucleotide
gtcagggttcagttagccaaaaaggagtaggggatcttttaaagaaaacaatcgatgctgtttccctgcaggaaagcgcccccaagagagaccatctcccgaagcagaggctctggctgta

SEQ ID NO: 43 - DNA, 91 bp, Artificial Sequence
  Synthetic oligonucleotide
tctctcttgggggcgctttcctgcagggaaacagcatcgattgttttctttaaaagatcccctactcctttttggctaactgaaccctgac

SEQ ID NO: 44 - DNA, 77 bp, Artificial Sequence
  Synthetic oligonucleotide
gtcagggttcagttagccaaaaaggagtaggggatcttttaaagaaaacaatcgatgctgtttccctgcaggaaagc

(19) United States
(12) Patent Application Publication
(10) Pub. No.: US 2023/0250470 A1
(43) Pub. Date: Aug. 10, 2023

(54) AMPLICON COMPREHENSIVE ENRICHMENT

(71) Applicant: William Marsh Rice University, Houston, TX (US)

(72) Inventors: David ZHANG, Houston, TX (US); Kerou ZHANG, Houston, TX (US); Ping SONG, Houston, TX (US)

(73) Assignee: William Marsh Rice University, Houston, TX (US)

(21) Appl. No.: 18/003,412

(22) PCT Filed: Jun. 25, 2021
(86) PCT No.: PCT/US21/39202

§ 371 Date: Dec. 27, 2022

Related U.S. Application Data
(60) Provisional application No. 63/044,634, filed on Jun. 26, 2020.

(51) Int. Cl. C12Q 1/6844; C12Q 1/6827; C12Q 1/6876

(52) U.S. Cl. CPC C12Q 1/6844; C12Q 1/6827; C12Q 1/6876; C12Q 2600/16; C12Q 2600/156

(57) ABSTRACT

Provided herein are reagents and methods for comprehensively enriching potential variants within targeted regions, named Amplicon Comprehensive Enrichment (ACE). The sequence variants enriched can include single nucleotide polymorphisms (SNPs), single nucleotide variants, or small insertions and deletions. Embodiments include procedures for integration with real-time polymerase chain reaction, next generation sequencing (NGS), and long-read sequencing.

### REFERENCE TO RELATED APPLICATIONS

The present application claims the priority benefit of U.S. provisional application No. 63/044,634, filed Jun. 26, 2020, the entire contents of which is incorporated herein by reference.

### STATEMENT REGARDING FEDERALLY SPONSORED RESEARCH

This invention was made with government support under Grant No. R01CA203964 awarded by the National Institutes of Health. The government has certain rights in the invention.

### REFERENCE TO A SEQUENCE LISTING

The instant application contains a Sequence Listing, which has been submitted in ASCII format via EFS-Web and is hereby incorporated by reference in its entirety. Said ASCII copy, created on Jun. 8, 2021, is named RICEP0077WO_ST25.txt and is 11.6 kilobytes in size.

### BACKGROUND

### 1. Field

The present disclosure relates generally to the field of molecular biology. More particularly, it concerns reagents and methods for comprehensively enriching potential variants within targeted regions, named Amplicon Comprehensive Enrichment (ACE).

### 2. Description of Related Art

Detection of DNA sequence variants with low variant allele fractions (VAFs) is critical for a range of research and clinical applications, including the detection of tumor-specific mutations in cancer tumor biopsy and plasma cell-free DNA samples. Current instruments for detection of low VAF mutations are either limited in sensitivity (e.g. Sanger and nanopore sequencing at 10% VAF) or limited in multiplexing (e.g. digital PCR at 1-plex) or expensive (e.g. deep sequencing to 25,000× or more with unique molecular identifier (UMI) barcodes). Selective enrichment of DNA sequence variants would improve the sensitivity of qPCR, Sanger sequencing, and nanopore sequencing and simultaneously decrease the cost of sequencing by synthesis (NGS).

Prior methods for variant enrichment, such as based on oscillating electric fields (Boreal Genomics), PCR with complex ramping (ICE-COLD PCR), nucleic acid analog blockers (Diacarta), and enzymatic digestion (Name-Pro) are typically difficult to productize for multiplexed panels, due to the high fragility of these methods to ambient temperature and probe sequence. The blocker displacement amplification method (Nuprobe) allows temperature-robust variant sequence enrichment, but has an enrichment window of about 20 nucleotides (nt) that renders it unwieldy for interrogating mutations in long continuous exons, such as in tumor suppressor genes like TP53 and BRCA1. Thus, new methods of variant enrichment that allow for interrogating mutations in long regions are needed.

### SUMMARY

As such, provided herein is Allele Comprehensive Enrichment (ACE) technology, which allows multiplexed variant allele enrichment in long regions ranging up to 100 nt.

In one embodiment, provided herein are compositions comprising: (a) an Auxiliary oligonucleotide, (b) a Suppressor oligonucleotide, wherein the Suppressor oligonucleotide comprises a Protected Subsequence that is at least 20 nucleotides long and that is reverse complementary to a subsequence of the Auxiliary oligonucleotide, wherein the Suppressor oligonucleotide comprises an Unprotected Subsequence that is at least 7 nucleotides long and that is not reverse complementary to the Auxiliary oligonucleotide, (c) a Forward Primer oligonucleotide, wherein the Forward Primer oligonucleotide comprises an at least 6 nucleotide long subsequence that is identical to a subsequence of the Suppressor oligonucleotide, and (d) a template-dependent polymerase. In some aspects, the compositions further comprise reagents and buffers needed for polymerase function. In some aspects, the template-dependent polymerase is a DNA polymerase. In some aspects, the template-dependent polymerase is a reverse transcriptase. In some aspects, the template-dependent polymerase is an RNA polymerase.

In some aspects, the compositions further comprise a nucleic acid Template molecule, wherein the Template molecule comprises a subsequence that is over 90% homologous to the reverse complement of the 3′ subsequence of the Forward Primer oligonucleotide. In some aspects, the Template molecule is a biological DNA or RNA molecule. In some aspects, the Template molecule is obtained from a sample of cells. In some aspects, the Template molecule is obtained from a biofluid. In certain aspects, the biofluid is blood, urine, saliva, cerebrospinal fluid, interstitial fluid, or synovial fluid. In some aspects, the Template molecule is obtained from a tissue. In certain aspects, the tissue is a biopsy tissue or a surgically resected tissue. In some aspects, the Template molecule is a complementary DNA molecule generated through the reverse transcription of an RNA molecule. In some aspects, the RNA molecule is obtained from a biological RNA sample derived from a human, animal, plant, or environmental specimen. In some aspects, the Template molecule is an amplicon DNA molecule generated through a DNA polymerase acting on a single-stranded DNA template. In some aspects, the Template molecule is an amplicon DNA molecule generated from multiple displacement amplification of a single cell DNA. In some aspects, the Template molecule is a physically, chemically, or enzymatically generated product of a biological DNA molecule. In some aspects, the Template molecule is the product of a fragmentation process. In some aspects, the fragmentation process is ultrasonication or enzymatic fragmentation. In some aspects, the Template molecule is the product of a bisulfite conversion reaction, an APOBEC reaction (i.e., a apolipoprotein B mRNA editing enzyme, catalytic polypeptide-like reaction), a TAPS reaction (i.e., TET-assisted pyridine borane sequencing reaction), or other chemical or enzymatic reaction in which cytosine nucleotides are selectively converted to uracil nucleotides based on its methylation status.

In some aspects, the Auxiliary oligonucleotide comprises an Initiation Complement Subsequence and a Target-binding Complement Subsequence. In some aspects, the Auxiliary oligonucleotide comprises DNA. In some aspects, the Auxiliary oligonucleotide consists of DNA. In some aspects, the Auxiliary oligonucleotide comprises non-natural oligonucleotides. In some aspects, the Auxiliary oligonucleotide has a length between 30 and 500 nt, 50 and 500 nt, 100 and 500 nt, 30 and 400 nt, 30 and 300 nt, 30 and 200 nt, 30 and 100 nt, and 75 nt, and 30 and 50 nt. In some aspects, the Auxiliary oligonucleotide is at least or about 30, 35, 40, 45, 50, 55, 60, 65, 70, 75, 80, 85, 90, 95, 100, 150, 200, 250, 300, 350, 400, 450, or 500 nucleotides long. In some aspects, the Auxiliary oligonucleotide has a 3′ chemical modification or DNA sequence that prevents DNA polymerase extension.

In some aspects, the Suppressor oligonucleotide comprises an Unprotected Subsequence and a Protected Subsequence. The Protected Subsequence consists of a Target-binding Subsequence and an Initiation Subsequence. In some aspects, the Protected Subsequence is at least 20 nucleotides long and is reverse complementary to at least a subsequence of the Auxiliary oligonucleotide. In some aspects, the Protected Subsequence is at least or about 20, 25, 30, 35, 40, 45, 50, 55, 60, 65, 70, 75, 80, 85, 90, 95, or 100 nucleotides long. In some aspects, the Protected Subsequence is between 20-500 nt, 20-400 nt, 20-300 nt, 20-200 nt, 20-100 nt, 20-80 nt, 20-60 nt, 30-500 nt, 30-400 nt, 30-300 nt, 30-200 nt, 30-100 nt, 30-80 nt, 30-60 nt, 40-500 nt, 40-400 nt, 40-300 nt, 40-200 nt, 40-100 nt, 40-80 nt, 50-500 nt, 50-400 nt, 50-300 nt, 50-200 nt, 50-100 nt, 50-80 nt, or 100-500 nt in length. In some aspects, the Protected Subsequence is reverse complementary to the entirety of the Auxiliary oligonucleotide. In some aspects, the Unprotected Subsequence is at least 7 nucleotides long and is not reverse complementary to any portion of the Auxiliary oligonucleotide. In some aspects, the Unprotected Subsequence is at least or about 8, 9, 10, 11, 12, 13, 14, 15, 16, 17, 18, 19, 20, 21, 22, 23, 24, 25, 26, 27, 28, 29, or 30 nucleotides long. In some aspects, the Unprotected Subsequence is between 8-30 nt, 8-25 nt, 8-20 nt, 8-15 nt, 12-30 nt, 12-25 nt, 12-20 nt, 16-30 nt, or 16-25 nt long.

In some aspects, the Initiation Subsequence is at or near the 3′ of the Suppressor oligonucleotide. In some aspects, the Initiation Subsequence has a length between 4 and 30 nucleotides (e.g., 4, 5, 6, 7, 8, 9, 10, 11, 12, 13, 14, 15, 16, 17, 18, 19, 20, 21, 22, 23, 24, 25, 26, 27, 28, 29, or 30 nucleotides; or between 4-30 nt, 4-25 nt, 4-20 nt, 4-15 nt, 8-30 nt, 8-25 nt, 8-20 nt, 8-15 nt, 12-30 nt, 12-25 nt, 12-20 nt, 16-30 nt, or 16-25 nt). In some aspects, the Initiation Subsequence is less than 30% identical (i.e., less than about 30%, 28%, 26%, 24%, 22%, 20%, 18%, or 16% identical) to the reverse complement of the Template molecule subsequence that is immediately to the 3′ of the Target Subsequence. In some aspects, the Auxiliary oligonucleotide has an Initiation Complement Subsequence at or near the 5′ end of the Auxiliary oligonucleotide. In some aspects, the Initiation Complement Subsequence has a length between 4 and 35 nucleotides (e.g., 4, 5, 6, 7, 8, 9, 10, 11, 12, 13, 14, 15, 16, 17, 18, 19, 20, 21, 22, 23, 24, 25, 26, 27, 28, 29, or 30 nucleotides; or between 4-30 nt, 4-25 nt, 4-20 nt, 4-15 nt, 8-30 nt, 8-25 nt, 8-20 nt, 8-15 nt, 12-30 nt, 12-25 nt, 12-20 nt, 16-30 nt, or 16-25 nt). In some aspects, the Initiation Complement Subsequence is at least 90% identical (e.g., about 90%, 91%, 92%, 93%, 94%, 95%, 96%, 97%, 98%, 99%, or 100% identical) to the reverse complement of the Initiation Subsequence of the Suppressor oligonucleotide. In some aspects, the Auxiliary oligonucleotide does not have a subsequence that is more than 30% identical (e.g., is not more than about 30%, 28%, 26%, 24%, 22%, 20%, 18%, or 16% identical) to the reverse complement of the Forward Primer oligonucleotide.

In some aspects, the Suppressor oligonucleotide comprises DNA. In some aspects, the Suppressor oligonucleotide consists of DNA. In some aspects, the Suppressor oligonucleotide comprises non-natural oligonucleotides. In some aspects, the Suppressor oligonucleotide has a length between 30 and 500, 50 and 500, 100 and 500, 30 and 400, 30 and 300, 30 and 200, 30 and 100, 30 and 75, and 30 and 50 nucleotides. In some aspects, the Suppressor oligonucleotide is at least or about 30, 35, 40, 45, 50, 55, 60, 65, 70, 75, 80, 85, 90, 95, 100, 150, 200, 250, 300, 350, 400, 450, or 500 nucleotides long. In some aspects, the Unprotected Subsequence of the Suppressor oligonucleotide is not reverse complementary to any portion of the Auxiliary oligonucleotide. In some aspects, the Suppressor oligonucleotide has a 3′ chemical modification or DNA sequence that prevents DNA polymerase extension.

In some aspects, the Forward Primer oligonucleotide comprises an at least 6 nucleotide long subsequence that is identical to a subsequence of the Suppressor oligonucleotide. In some aspects, the Forward Primer oligonucleotide comprises DNA. In some aspects, the Forward Primer oligonucleotide consists of DNA. In some aspects, the Forward Primer oligonucleotide comprises RNA. In some aspects, the Forward Primer oligonucleotide consists of RNA. In some aspects, the Forward Primer oligonucleotide has a length between 6 and 70 nucleotides (e.g., 6, 7, 8, 9, 10, 11, 12, 13, 14, 15, 16, 17, 18, 19, 20, 21, 22, 23, 24, 25, 26, 27, 28, 29, 30, 35, 40, 45, 50, 55, 60, 65, or 70 nucleotides; or between 6-70 nt, 6-30 nt, 6-25 nt, 6-20 nt, 6-15 nt, 8-70 nt, 8-30 nt, 8-25 nt, 8-20 nt, 8-15 nt, 12-70 nt, 12-30 nt, 12-25 nt, 12-20 nt, 16-70 nt, 16-30 nt, or 16-25 nt). In some aspects, the Forward Primer oligonucleotide and the Auxiliary oligonucleotide are not able to hybridize with each other.

In some aspects, the compositions further comprise a Reverse Primer oligonucleotide, wherein the Template molecule comprises a subsequence that is over 90% homologous (e.g., about 90%, 91%, 92%, 93%, 94%, 95%, 96%, 97%, 98%, 99%, or 1090% homologous) to a 3′ subsequence of the Reverse Primer oligonucleotide. In some aspects, the Reverse Primer oligonucleotide has a length between 10 and 70 nucleotides (e.g., 10, 11, 12, 13, 14, 15, 16, 17, 18, 19, 20, 21, 22, 23, 24, 25, 26, 27, 28, 29, 30, 35, 40, 45, 50, 55, 60, 65, or 70 nucleotides; or between 10-70 nt, 10-30 nt, 10-25 nt, 10-20 nt, 10-15 nt, 12-70 nt, 12-30 nt, 12-25 nt, 12-20 nt, 16-70 nt, 16-30 nt, or 16-25 nt).

In some aspects, the Template molecule comprises a Target Subsequence positioned between a Forward Primer-binding Subsequence and a Reverse Primer-homologous Subsequence. In some aspects, the Target Subsequence is at least 70% identical (e.g., about 70%, 75%, 80%, 85%, 90%, 91%, 92%, 93%, 94%, 95%, 96%, 97%, 98%, 99%, or 100% identical) to the reverse complement of the portion of Suppressor oligonucleotide Protected Subsequence that does not include the Initiation Subsequence, i.e., the Target-binding Subsequence.

In some aspects, the Suppressor oligonucleotide and the Auxiliary oligonucleotide each have a 3′ chemical modification that prevents DNA polymerase extension. In some aspects, the modification comprises dideoxynucleotides, inverted DNA nucleotides, phosphonothioate-substituted backbone, and alkane or polyethylene glycol (PEG) spacers.

In some aspects, the Suppressor oligonucleotide and the Auxiliary oligonucleotide each have a DNA sequence that prevents DNA polymerase extension. In some aspects, the DNA sequence at the 3′ end forms at least one hairpin structure.

In some aspects, the compositions further comprise a fluorophore-functionalized DNA probe. In certain aspects, the fluorophore-functionalized DNA probe is a Taqman probe or a molecular beacon.

In some aspects, the compositions further comprise a DNA intercalating dye. In certain aspects, the DNA intercalating dye is SybrGreen, EvaGreen, or Syto.

In some aspects, the stoichiometric ratio of the Auxiliary oligonucleotide to the Suppressor oligonucleotide is between 0.8 and 100.

In some aspects, the Forward Primer oligonucleotide and the Template molecule have a standard free energy of hybridization (ΔG°₁) between −7.0 kcal/mol and −20.0 kcal/mol at a temperature of 60° C. and a salinity of 0.2 M sodium. For example, the Forward Primer oligonucleotide and the Template molecule have a standard free energy of hybridization (ΔG°₁) between −7.0 and −20.0 kcal/mol, between −7.0 and −18.0 kcal/mol, between −7.0 and −16.0 kcal/mol, between −7.0 and −14.0 kcal/mol, between −7.0 and −12.0 kcal/mol, between −7.0 and −10.0 kcal/mol, between −10.0 and −20.0 kcal/mol, between −10.0 and −18.0 kcal/mol, between −10.0 and −16.0 kcal/mol, between-10.0 and −14.0 kcal/mol, or any range derivable therein. In some aspects, the Forward Primer oligonucleotide and the Template molecule have a standard free energy of hybridization (ΔG°₁) that is at least or about −7.0, −8.0, −9.0, −10.0, −11.0, −12.0, −13.0, −14.0, −15.0, −16.0, −17.0, −18.0, −19.0, or −20.0 kcal/mol.

In some aspects, the Suppressor oligonucleotide and the Template molecule have a standard free energy of hybridization (ΔG°₂) between −16 kcal/mol and −200 kcal/mol at a temperature of 60° C. and a salinity of 0.2 M sodium. For example, the Suppressor oligonucleotide and the Template molecule have a standard free energy of hybridization (ΔG°₂) between −16 and −200 kcal/mol, between −16 and −150 kcal/mol, between −16 and −100 kcal/mol, between −16 and −50 kcal/mol, between −16 and −25 kcal/mol, between −25 and −200 kcal/mol, between −25 and −150 kcal/mol, between −25 and −100 kcal/mol, between −25 and −75 kcal/mol, between −25 and −50 kcal/mol. In some aspects, the Suppressor oligonucleotide and the Template molecule have a standard free energy of hybridization (ΔG°₂) that is at least or about −16, −18, −20, −25, −30, −35, −40, −45, −50, −55, −60, −65, −70, −75, −80, −85, −90, −95, −100, −125, −150, −175, or −200 kcal/mol.

In some aspects, the Suppressor oligonucleotide and the Auxiliary oligonucleotide have a standard free energy of hybridization (ΔG°₃) between −15 kcal/mol and −200 kcal/mol at a temperature of 60° C. and a salinity of 0.2 M sodium. For example, the Suppressor oligonucleotide and the Auxiliary oligonucleotide have a standard free energy of hybridization (ΔG°₃) between −15 and −200 kcal/mol, between −15 and −150 kcal/mol, between −15 and −100 kcal/mol, between −15 and −50 kcal/mol, between −15 and −25 kcal/mol, between −25 and −200 kcal/mol, between −25 and −150 kcal/mol, between −25 and −100 kcal/mol, between −25 and −75 kcal/mol, between −25 and −50 kcal/mol. In some aspects, the Suppressor oligonucleotide and the Auxiliary oligonucleotide have a standard free energy of hybridization (ΔG°₃) that is at least or about −15, −16, −17, −18, −19, −20, −25, −30, −35, −40, −45, −50, −55, −60, −65, −70, −75, −80, −85, −90, −95, −100, −125, −150, −175, or −200 kcal/mol.

In some aspects, the value of (ΔG°₂−ΔG°₃) is between −5 kcal/mol and +5 kcal/mol. For example, the value of (ΔG°₂−ΔG°₃) is between −5 and +5 kcal/mol, between −5 and +4 kcal/mol, between −5 and +3 kcal/mol, between −5 and +2 kcal/mol, between −5 and +1 kcal/mol, between −5 and 0 kcal/mol, between −5 and −1 kcal/mol, between −5 and −2 kcal/mol, between −5 and −3 kcal/mol, between −4 and +5 kcal/mol, between −3 and +5 kcal/mol, between −2 and +5 kcal/mol, between −1 and +5 kcal/mol, between 0 and +5 kcal/mol, between +1 and +5 kcal/mol, +2 and +5 kcal/mol, between +3 and +5 kcal/mol, between −4 and +4 kcal/mol, between −3 and +3 kcal/mol, between −2 and +2 kcal/mol, between −1 and +1 kcal/mol, between −2 and 0 kcal/mole, between −2 and +1 kcal/mol, or between −1 and +2 kcal/mol. In some aspects, the value of (ΔG°₂−ΔG°₃) is at least or about −5, −4, −3, −2, −1, 0 +1, +2, +3, +4, or +5 kcal/mol.

In some aspects, the Reverse Primer oligonucleotide and the variant Template molecule have a standard free energy of hybridization (ΔG°₄) between −7 kcal/mol and −20 kcal/mol at a temperature of 60° C. and a salinity of 0.2 M sodium. For example, the Reverse Primer oligonucleotide and the Template molecule have a standard free energy of hybridization (ΔG°₄) between −7 and −20 kcal/mol, between −7 and −18 kcal/mol, between −7 and −16 kcal/mol, between −7 and −14 kcal/mol, between −7 and −12 kcal/mol, between −7 and −10 kcal/mol, between −10 and −20 kcal/mol, between −10 and −18 kcal/mol, between −10 and −16 kcal/mol, or between −10 and −14 kcal/mol. In some aspects, the Reverse Primer oligonucleotide and the Template molecule have a standard free energy of hybridization (ΔG°₄) that is at least or about −7, −8, −9, −10, −11, −12, −13, −14, −15, −16, −17, −18, −19, or −20 kcal/mol.

In some aspects, the Suppressor oligonucleotide hybridizing to the wildtype DNA Template molecule is more thermodynamically favorable than the Suppressor oligonucleotide binding to the Auxiliary oligonucleotide, which is more thermodynamically favorable than the Forward Primer oligonucleotide binding to the DNA Template molecule, which is more thermodynamically favorable than the Suppressor oligonucleotide binding to the variant DNA Template molecule.

In some aspects, the composition comprises a plurality of Suppressor oligonucleotide species, a plurality of Auxiliary oligonucleotide species, and a plurality of Forward Primer oligonucleotide species. In some aspects, each Suppressor oligonucleotide species comprises a Protected Subsequence that is at least 20 nucleotides long and that is reverse complementary to a subsequence of at least one corresponding Auxiliary oligonucleotide species. In some aspects, each Forward Primer oligonucleotide species comprises an at least 6 nucleotide long subsequence that is identical to a subsequence of at least one corresponding Suppressor oligonucleotide species. In some aspects, the plurality of Forward Primer oligonucleotide species each comprises a first universal Adapter sequence at its 5′ region.

In one embodiment, provided herein are methods for selectively amplifying a DNA sequence variant using polymerase chain reaction, the methods comprising: (a) mixing a Sample possibly comprising a variant DNA Template molecule and possibly comprising a wildtype DNA Template molecule a composition of any of the present embodiment, and (b) subjecting the mixture to at least 7 rounds of thermal cycling. For example, the thermal cycling may be performed for at least or about 7, 8, 9, 10, 11, 12, 13, 14, 15, 16, 17, 18, 19, 20, 25, 30, 35, or 40 cycles. In some aspects, each round of thermal cycling comprises holding the mixture at a denaturing temperature of between 80° C. and 105° C. (e.g., between 80 and 105° C., between 80 and 100° C., between 80 and 95° C., between 85 and 105° C., between 85 and 100° C., between 85 and 95° C.; or at least or about 80, 85, 90, 95, 100, or 105° C.) for between 1 second and 1 hour (e.g., between 1 second-1 hour, between 1 second-30 minutes, seconds-30 minutes, 20 seconds-30 minutes, 30 seconds-30 minutes, 45 seconds-30 minutes, 1 minute-30 minutes, 2-minutes-30 minutes, 30 second-5 minutes, or 1 minute-5 minutes; or at least or about 1 second, 5 seconds, 10 seconds, 15 seconds, 20 seconds, 30 seconds, 45 seconds, 1 minutes, 2 minutes, 5 minutes, or 10 minutes) and then holding the mixture at an annealing temperature of between 50° C. and 75° C. (e.g., between 50 and 75° C., between 50 and 72° C., between 50 and 70° C., between 50 and 65° C., between 50 and 60° C., between 55 and 75° C., between 55 and 72° C., between 55 and 70° C., between 55 and 65° C., between 60 and 75° C., between 60 and 72° C., between 60 and 70° C., between 65 and 75° C.; or at least or about 50, 55, 60, 65, 70, or 75° C.) for between 1 second and 2 hours (e.g. between 1 second-2 hours, 1 second-1 hour, 1 second-30 minutes, 10 seconds-30 minutes, 20 seconds-30 minutes, 30 seconds-30 minutes, 45 seconds-30 minutes, 1 minute-30 minutes, 2-minutes-30 minutes, 30 second-5 minutes, or 1 minute-5 minutes; or at least or about 1 second, 5 seconds, 10 seconds, 15 seconds, 20 seconds, 30 seconds, 45 seconds, 1 minutes, 2 minutes, 5 minutes, or 10 minutes).

In some aspects, a plurality of Forward Primer oligonucleotides, Reverse Primer oligonucleotides, Suppressor oligonucleotides, and Auxiliary oligonucleotides are mixed with the Sample, wherein each set of Forward Primer oligonucleotides, Reverse Primer oligonucleotides, Suppressor oligonucleotides, and Auxiliary oligonucleotides corresponds to different variant Template molecule and wildtype Template molecule sequences. In some aspects, all Forward Primer oligonucleotides comprise a Universal Forward Adapter subsequence at or near the 5′ end, and wherein all Reverse Primer oligonucleotides comprise a Universal Reverse Adapter subsequence at or near the 5′ end. In some aspects, each Suppressor oligonucleotide species comprises a Protected Subsequence that is at least 20 nucleotides long and that is reverse complementary to a subsequence of at least one corresponding Auxiliary oligonucleotide species. In some aspects, each Forward Primer oligonucleotide species comprises an at least 6 nucleotide long subsequence that is identical to a subsequence of at least one corresponding Suppressor oligonucleotide species.

In some aspects, the concentration of each Forward Primer oligonucleotide in the mixture is between 100 pM and 5 μM. In some aspects, the concentration of each Reverse Primer oligonucleotide in the mixture is between 100 pM and 5 μM. In some aspects, the concentration of each Suppressor oligonucleotide in the mixture is between 100 pM and 5 μM. In some aspects, the concentration of each Auxiliary oligonucleotide is between 100 pM and 5 μM. For any of these, the concentration may be between 100 pM-5 μM, 200 pM-5 μM, 300 pM-5 μM, 400 pM-5 μM, 500 pM-5 μM, 750 pM-5 μM, 1 nM-5 μM, 250 nM-5 μM, 500 nM-5 μM, 750 nM-5 μM, 1 μM-5 μM, 100 pM-1 μM, 200 pM-1 μM, 300 pM-1 μM, 400 pM-1 μM, 500 pM-1 μM, 750 pM-1 μM, 1 nM-1 μM, or 500 pM-500 nM. For example, the concentration may be at least or about 100 pM, 200 pM, 300 pM, 400 pM, 500 pM, 750 pM, 1 nM, 10 nM, 50 nM, 100 nM, 200 nM, 300 nM, 400 nM, 500 nM, 750 nM, 1 μM, 2 μM, 3 μM, 4 μM, or 5 μM.

In some aspects, the stoichiometric ratio of each Forward Primer oligonucleotide to its corresponding Suppressor oligonucleotide is between 0.8 and 100. In some aspects, the stoichiometric ratio of each Auxiliary oligonucleotide to its corresponding Suppressor oligonucleotide is between 0.8 and 100.

In some aspects, the mixture further comprises a fluorophore-functionalized DNA probe. In certain aspects, the fluorophore-functionalized DNA probe is a Taqman probe or a molecular beacon. In some aspects, the mixture further comprises a DNA intercalating dye. In certain aspects, the DNA intercalating dye is SybrGreen, EvaGreen, or Syto.

In one embodiment, provided herein are methods for selectively detecting and quantifying DNA sequence variants using quantitative PCR (qPCR), the methods comprising: (a) performing selective PCR amplification of variant DNA templates over wildtype DNA templates in a first aliquot of a Sample according to the selective amplification methods of any one of the present embodiments; (b) performing time-based measurements of solution fluorescence; (c) calculating a cycle threshold (Ct) value based on the cycle in which the solution fluorescence exceeds a threshold; and (d) making a determination of the presence/absence or quantity of the variant DNA template in the Sample based on the Ct value. In some aspects, the qPCR mixture comprises a Taqman probe. In some aspects, the methods further comprise (e) performing a second qPCR reaction on a second aliquot of the Sample using the Forward Primer oligonucleotide and the Reverse Primer oligonucleotide, in the absence of Suppressor oligonucleotide; (f) calculating a cycle threshold (Ct2) of this second reaction; and (g) making a determination on the relative quantity of variant DNA Template to wildtype DNA Template based on the difference in values between Ct and Ct2.

In one embodiment, provided herein are methods for selectively detecting and quantifying DNA sequence variants using high-throughput sequencing, the method comprising: (a) performing selective PCR amplification of variant DNA templates over wildtype DNA templates in a first aliquot of a Sample according to the selective amplification methods of any one of the present embodiments; and (b) performing high-throughput sequencing on the PCR product of step (a).

In some aspects, the Forward Primer oligonucleotide comprises a forward sequencing adapter at its 5′ end, and the Reverse Primer oligonucleotide comprises a reverse sequencing adapter at its 5′ end. In certain aspects, one or both of the sequencing adapters comprise unique molecular identifier (UMI) sequences.

In some aspects, the methods further comprise appending sequencing adapters and/or sequencing indexes using PCR. In certain aspects, the sequencing adapters comprise unique molecular identifier (UMI) sequences.

In some aspects, the methods further comprise ligating a sequencing adapter to the PCR product of step (a) before performing high-throughput sequencing. In certain aspects, the sequencing adapters appended via ligation comprise unique molecular identifier (UMI) sequences.

In some aspects, the UMI sequences comprise a set of pre-designed sequences wherein every pair of UMI sequences exhibit a minimal Hamming distance that is not less than 30% of the length of the UMI. In some aspects, the UMI sequences comprise a set of sequences comprising degenerate nucleotides, selected from N (mixture of A, C, G, and T), B (mixture of C, G, and T), D (mixture of A, G, and T), H (mixture of C, A, and T), V (mixture of A, C, and G), S (mixture of C and G), W (mixture of A and T), R (mixture of A and G), Y (mixture of T and C), K (mixture of G and T), and M (mixture of A and C).

In some aspects, the high-throughput sequencing is performed via sequencing-by-synthesis. In some aspects, the high-throughput sequencing is performed via electrical current measurements in conjunction with a nanopore.

Other objects, features and advantages of the present invention will become apparent from the following detailed description. It should be understood, however, that the detailed description and the specific examples, while indicating preferred embodiments of the invention, are given by way of illustration only, since various changes and modifications within the spirit and scope of the invention will become apparent to those skilled in the art from this detailed description.

### BRIEF DESCRIPTION OF THE DRAWINGS

The following drawings form part of the present specification and are included to further demonstrate certain aspects of the present invention. The invention may be better understood by reference to one or more of these drawings in combination with the detailed description of specific embodiments presented herein.

FIG. 1: Key reagent components for the Amplicon Comprehensive Enrichment (ACE) system. The arrows on the right side of the Forward Primer oligonucleotides denote the 3′ ends of the oligonucleotides. The vertical bar on the left side of the Auxiliary oligonucleotide and the diagonal bar on the right side of the Suppressor oligonucleotide denote the 3′ ends of the oligonucleotides and further denotes that there is a chemical modification or DNA sequence that prevents polymerase extension. The Suppressor oligonucleotide and the Auxiliary oligonucleotide have a significant region of reverse complementarity, defined as the Protected Subsequence and illustrated as regions where the two oligos are placed close to each other. The Forward Primer oligonucleotide and the Suppressor oligonucleotide have a significant region of sequence similarity, illustrated as the horizontal positions of the Forward Primer oligonucleotide and the Suppressor oligonucleotide. The ACE system also includes a template-dependent polymerase and dNTP reagents needed for the polymerase to perform primer extension.

FIG. 2: Application of ACE in polymerase chain reaction (PCR)-based enrichment of variant alleles. The Suppressor oligonucleotide preferentially binds to the Template molecule both because it is longer and has additional sites for initiation of hybridization, and because it may be present at a higher concentration than the Forward Primer oligonucleotide. While the Suppressor oligonucleotide is bound to the Template molecule, the Forward Primer oligonucleotide cannot hybridize to the Template molecule, and thus cannot be extended by the DNA polymerase. The Initiation Subsequence on the Suppressor oligonucleotide can bind to the Initiation Complement Subsequence on the Auxiliary oligonucleotide and branch migration can begin. However, on a Template molecule matched to the Suppressor oligonucleotide in the Target Subsequence, the displacement of the Suppressor oligonucleotide by the Auxiliary oligonucleotide is unlikely to occur due to either thermodynamics or the kinetics given limited PCR anneal cycle times. The Reverse Primer oligonucleotide is a standard PCR reverse primer and does not necessarily have any sequence similarity or reverse complementarity to any of the other oligonucleotides.

FIG. 3: Application of ACE in PCR-based enrichment of variant alleles. On a Template molecule with a sequence variant, the mismatch bubble formed between the Template molecule and the Suppressor oligonucleotide in the Target Subsequence thermodynamically destabilizes the Template-Suppressor hybridization and makes the displacement of the Suppressor oligonucleotide by the Auxiliary oligonucleotide more thermodynamically favorable. Furthermore, the mismatch bubble represents a kinetic trap in the displacement reaction that also speeds up the kinetics. After the Suppressor oligonucleotide is displaced by the Auxiliary oligonucleotide, the Template molecule is free to bind to the Forward Primer oligonucleotide, and the Forward Primer oligonucleotide is subsequently extended as in standard PCR.

FIG. 4: Detailed example of an ACE mixture intended to selectively PCR amplify one of two single nucleotide polymorphism (SNP) alleles at the rs1443486 SNP locus. The NA18562 human genomic DNA is homozygous for the A allele, and the NA18537 human genomic DNA is homozygous for the C allele on the (−) strand of DNA. A Taqman probe binds downstream of the Suppressor oligonucleotide to produce specific fluorescence signal for the amplicons generated. Here, the Suppressor oligonucleotide is designed to perfectly match the NA18562 A allele.

FIG. 5: Experimental results for ACE quantitative PCR (qPCR), using human genomic DNA. The cycle threshold (Ct) value of the qPCR reaction can be clearly distinguished between 100% NA18537, 5% NA18537/95% NA18562, 1% NA18537/99% NA18562, and 100% NA18562. In all reactions, 7.5 ng of human genomic DNA input was used, corresponding to approximately 2250 haploid genome copies. Higher concentrations of Suppressor oligonucleotide led to delayed Ct values for all DNA samples. All reactions used a PowerUp DNA polymerase mastermix.

FIG. 6: Further experimental results supporting the proposed ACE mechanism. Without either Suppressor oligonucleotide or Auxiliary oligonucleotide in the qPCR reaction, the Ct values of NA18537 and NA18562 are nearly identical, demonstrating that the input quantities are similar and PCR efficiencies are similar. With only Auxiliary oligonucleotide but not Suppressor oligonucleotide, the Ct values are identical to the qPCR reactions with neither Auxiliary oligonucleotide nor Suppressor oligonucleotide, suggesting that Auxiliary oligonucleotide by itself does not inhibit the Reverse Primer oligonucleotide binding to the reverse complement of the Template molecule. With only Suppressor oligonucleotide but not Auxiliary oligonucleotide, there is no observable PCR amplification, suggesting that the Suppressor oligonucleotide completely inhibits Forward Primer oligonucleotide binding and PCR when it is not displaced by Auxiliary oligonucleotide. When both Suppressor oligonucleotide and Auxiliary oligonucleotide are present, then we observe preferential amplification of the NA18537, because its Template molecule is mismatched against the Suppressor oligonucleotide.

FIG. 7: Demonstration of comprehensiveness of ACE in qPCR settings. ACE qPCR was tested on 15 separate DNA sequences corresponding to TP53 mutations at different loci. Based on the design of the ACE mechanism, all mutations are selectively enriched by the same ACE Suppressor oligonucleotide and Auxiliary oligonucleotide regardless of the mutation's position on the Suppressor oligonucleotide. The experiment was performed using synthetic gBlock oligonucleotide templates (606 nt long each) as mutant Template and using NA18537 as wildtype Template. Plotted are the median Ct values of 3 triplicate reactions for mutations, and the Ct values of NA18537 gDNA are plotted as 3 horizontal lines. The Ct values of all TP53 mutations were significantly smaller than that of the NA18537 wildtype template alone, suggesting that all of these mutations were enriched.

FIG. 8: Demonstration that ACE functions for long Suppressor oligonucleotide and Auxiliary oligonucleotide. Three separate ACE systems were tested for the same rs1443486 SNP. The Suppressor oligonucleotides were designed to be varying lengths, with the length of the Template-binding region being 64 nt, 81 nt, and 126 nt. The SNP position was designed to be consistently the 13th nucleotide from the end of the Template-binding region. All three ACE systems showed significant Ct difference between the NA18537 template and the NA18562 template. Significant delay was observed for the longest Suppressor oligonucleotide, suggesting either that that longer length necessitate a longer anneal cycle time to allow strand displacement, or that the longer lengths causes the Auxiliary oligonucleotide purity to drop, rendering displacement less efficient.

FIG. 9: Application of ACE using Forward Primer oligonucleotide and Reverse Primer oligonucleotide with 5′ universal adapter sequences. The adapter sequences allow subsequent adapter PCR for next-generation sequencing (NGS) library preparation.

FIG. 10: Demonstration of highly multiplexed ACE using NGS. An 18-plex ACE panel, targeting 18 different SNP loci in which NA18537 and NA18562 were homozygous for different alleles, was constructed. All 18 Suppressor oligonucleotides were designed to be perfectly matched against the NA18562 alleles. The 18-plex ACE was tested on a sample of 1% NA18537/99% NA18562; each library used 25 ng of this mixture as input. In the absence of ACE, the NGS library showed that for each locus, the number of reads mapped to the NA18562 allele was roughly 100-fold higher than that of the NA18537 allele, as expected. With the 18-plex Adaptor ACE system (right), the fraction of NA18537 alleles at each locus was increased (note different Y-axis scale). The bottom table summarizes the NGS library results for 5 different libraries; the left-most and right-most libraries were plotted above. The VRF row refers to the Variant Allele Fraction, calculated as the number of variant (NA18537) reads divided by the sum of the variant reads and the wildtype (NA18562) reads.

FIG. 11: Embodiment of ACE in which multiple Suppressor oligonucleotides and Forward Primer oligonucleotides are tiled across a longer PCR amplicon. This can overcome the technical difficulties in the synthesis of very long Suppressor oligonucleotides, to enable the detection of variants in a broad region of many nucleotides in a single PCR amplicon.

FIG. 12: Embodiment of ACE used to enrich for gene fusion variants. Here, the gene fusion Template differs from the wildtype Template in possessing a downstream sequence from a different gene (zigzag line). The gene fusion variant may possibly be derived from abnormal chromosomal rearrangement, for example in a cancer cell. Importantly, the downstream fusion gene and sequence are not a priori known, so standard PCR-based ACE will not work because the Reverse Primer oligonucleotide sequence cannot be designed to target the downstream gene. Here, a double-stranded DNA adapter is ligated to the DNA templates, and the Reverse Primer oligonucleotide is designed to target the adapter sequence. The Suppressor oligonucleotide binds strongly to the wildtype Template and is not displaced by the Auxiliary oligonucleotide with high efficiency. The Suppressor oligonucleotide binds less strongly to the gene fusion Template, and is effectively displaced by the Auxiliary oligonucleotide, allowing the forward primer to bind to the gene fusion Template and amplify the Template.

FIG. 13: Embodiment of ACE on rolling circle amplification (RCA). Here, circular DNA Template molecules with wildtype or mutant sequence are differentially amplified via RCA because the Suppressor oligonucleotide binds strongly to the wildtype Template molecule and is not displaced by the Auxiliary oligonucleotide. The mismatch bubble formed between the Suppressor oligonucleotide and the mutant Template molecule destabilizes the binding between the Suppressor oligonucleotide and the mutant Template molecule, and allows the Auxiliary oligonucleotide to displace the Suppressor oligonucleotide, in turn allowing the Forward Primer oligonucleotide to bind and amplify the Template molecule. Note that in RCA, there is no Reverse Primer oligonucleotide.

### DETAILED DESCRIPTION

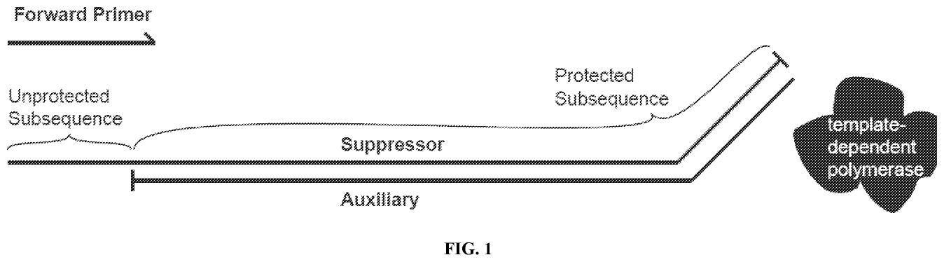

Allele Comprehensive Enrichment (ACE) is based on the design of a Suppressor oligonucleotide that exhibits significant 5′ sequence similarity to a corresponding Forward Primer oligonucleotide (FIG. 1). An Auxiliary oligonucleotide that exhibits significant reverse complementarity to the Suppressor oligonucleotide at the latter's 3′ end is used to inhibit the Suppressor oligonucleotide and allow efficient amplification of a nucleic acid variant Template molecule having a sequence that differs from an intended nucleic acid wildtype Template molecule. In some embodiments, the Template molecule is a DNA molecule. In other embodiments, the Template molecule is an RNA molecule.

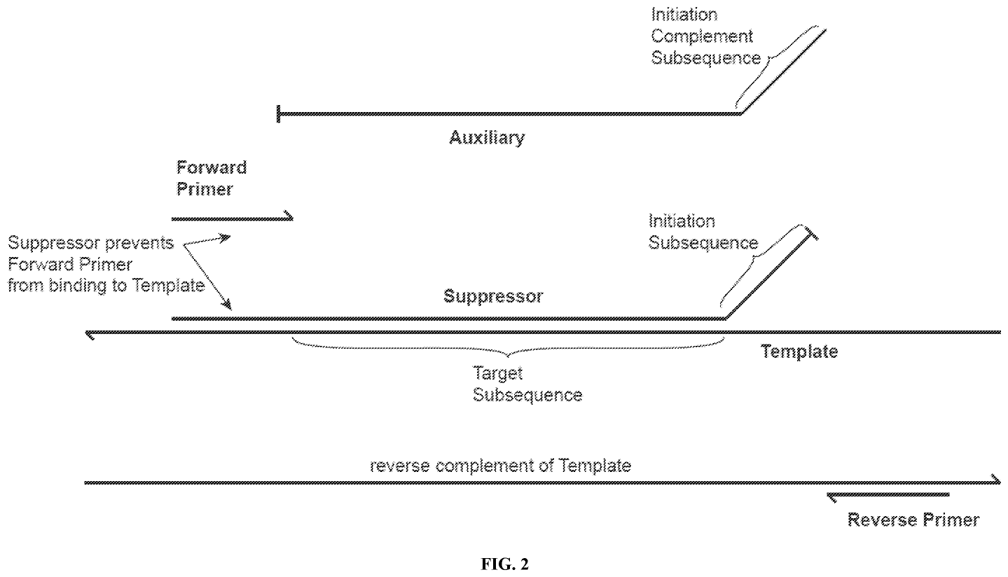

The Suppressor oligonucleotide has a sequence that is designed to bind more favorably to an intended wildtype Template sequence than it does to the Auxiliary oligonucleotide (FIG. 2). While the Suppressor oligonucleotide is bound to the Template molecule, the Forward Primer oligonucleotide cannot efficiently bind to the Template molecule, because part of the Template sequence that binds to the Suppressor oligonucleotide is also the subsequence that binds to the Forward Primer oligonucleotide. In some embodiments, the subsequence of the Template molecule that the Forward Primer oligonucleotide binds to is entirely encompassed within the subsequence of the Template molecule that the Suppressor oligonucleotide binds to. In other embodiments, the subsequence of the Template molecule that the Forward Primer oligonucleotide binds to has a small number of nucleotides, not larger than 7 nucleotides (i.e., 1, 2, 3, 4, 5, 6, or 7 nucleotide(s)), that is not encompassed within the subsequence of the Template molecule that the Suppressor oligonucleotide binds to.

The Suppressor oligonucleotide comprises the Initiation Subsequence, which is not reverse complementary to the Template molecule. The Auxiliary oligonucleotide comprises a Initiation Complement Subsequence, which is reverse complementary to the Initiation Sequence. In some embodiments, the Initiation Subsequence has a length between 4 nt and 30 nt (e.g., 4, 5, 6, 7, 8, 9, 10, 11, 12, 13, 14, 15, 16, 17, 18, 19, 20, 21, 22, 23, 24, 25, 26, 27, 28, 29, or 30 nucleotides; or between 4-30 nt, 4-25 nt, 4-20 nt, 4-15 nt, 8-30 nt, 8-25 nt, 8-20 nt, 8-15 nt, 12-30 nt, 12-25 nt, 12-20 nt, 16-30 nt, or 16-25 nt).

The region of the Template molecule that the Suppressor oligonucleotide binds to, that the Forward Primer oligonucleotide does not bind to, is known as the Target Subsequence. Sequence variations in the Target Subsequence will be preferentially amplified through ACE. In some embodiments, the Target Subsequence has a length between 10 nt and 500 nt. In other embodiments, the Target Subsequence has a length between 10 nt and 200 nt. For example, the Target Subsequence has a length between 10-500 nt, 10-400 nt, 10-300 nt, 10-200 nt, 10-100 nt, 10-80 nt, 10-60 nt, 10-40 nt, 20-500 nt, 20-400 nt, 20-300 nt, 20-200 nt, 20-100 nt, 20-80 nt, 20-60 nt, 30-500 nt, 30-400 nt, 30-300 nt, 30-200 nt, 30-100 nt, 30-80 nt, 30-60 nt, 40-500 nt, 40-400 nt, 40-300 nt, 40-200 nt, 40-100 nt, 40-80 nt, 50-500 nt, 50-400 nt, 50-300 nt, 50-200 nt, 50-100 nt, 50-80 nt, or 100-500 nt. For example, the Target Subsequence has a length of at least or about 10, 15, 20, 25, 30, 35, 40, 45, 50, 60, 70, 80, 90, 100, 120, 140, 160, 180, 200, 220, 240, 260, 280, 300, 320, 340, 360, 380, 400, 420, 440, 460, 480, or 500 nucleotides.

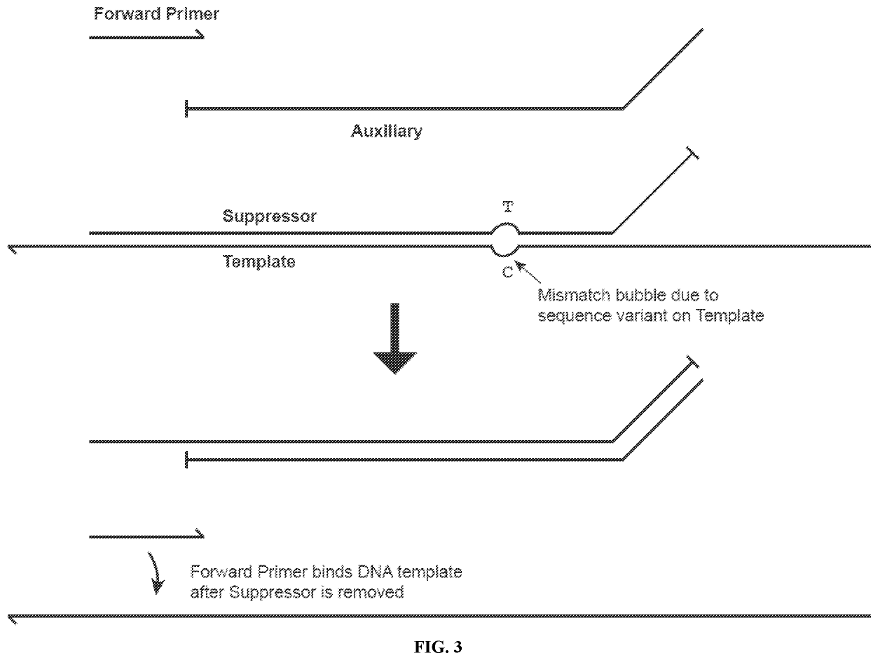

If the Template sequence has even a single nucleotide sequence variant, the mismatch bubble formed between the Template molecule and the Suppressor oligonucleotide in the Target Subsequence causes a thermodynamic destabilization that results in the Suppressor oligonucleotide binding more favorably to the Auxiliary oligonucleotide than to the Template molecule (FIG. 3). Consequently, the Suppressor oligonucleotide is displaced from the Template molecule, and the Template molecule is subsequently capable of binding to the Forward Primer oligonucleotide. In some embodiments, the Forward Primer oligonucleotide is then able to be extended by a template-dependent polymerase. In some embodiments, a mixture of wildtype Template molecules and variant Template molecules are present in a Template sample, and the application of ACE to the sample results in the enrichment of the variant Template molecules over the wildtype Template molecules through selective amplification of the variant Template molecules. In some embodiments, the template-dependent polymerase is a DNA polymerase. In some embodiments, the DNA polymerase is a thermostable DNA polymerase, and the amplification is achieved through polymerase chain reaction (PCR). In other embodiments, the DNA polymerase is a phi-29 polymerase, and the amplification is achieved through rolling circle amplification. In other embodiments, the template-dependent polymerase is a reverse transcriptase, and the enrichment of the mutant RNA Template over the wildtype RNA Template is through the selective reverse transcription of mutant RNA Templates.

### I. Definitions

“Amplification,” as used herein, refers to any in vitro process for increasing the number of copies of a nucleotide sequence or sequences. Nucleic acid amplification results in the incorporation of nucleotides into DNA or RNA. As used herein, one amplification reaction may consist of many rounds of DNA replication. For example, one PCR reaction may consist of 2-100 “cycles” of denaturation and replication.

“Polymerase chain reaction,” or “PCR,” means a reaction for the in vitro amplification of specific DNA sequences by the simultaneous primer extension of complementary strands of DNA. In other words, PCR is a reaction for making multiple copies or replicates of a target nucleic acid flanked by primer binding sites, such reaction comprising one or more repetitions of the following steps: (i) denaturing the target nucleic acid, (ii) annealing primers to the primer binding sites, and (iii) extending the primers by a nucleic acid polymerase in the presence of nucleoside triphosphates. Usually, the reaction is cycled through different temperatures optimized for each step in a thermal cycler instrument. In some cases, the annealing and extension steps may be combined into a single step. Particular temperatures, durations at each step, and rates of change between steps depend on many factors well-known to those of ordinary skill in the art, e.g., exemplified by the references: McPherson et al., editors, PCR: A Practical Approach and PCR2: A Practical Approach (IRL Press, Oxford, 1991 and 1995, respectively).

“Primer” means an oligonucleotide, either natural or synthetic that is capable, upon forming a duplex with a polynucleotide template, of acting as a point of initiation of nucleic acid synthesis and being extended from its 3′ end along the template so that an extended duplex is formed. The sequence of nucleotides added during the extension process is determined by the sequence of the template polynucleotide. Usually primers are extended by a DNA polymerase. Primers are generally of a length compatible with its use in synthesis of primer extension products, and are usually are in the range of between 6 to 100 nucleotides in length, such as 6 to 70, 10 to 50, 10 to 75, 15 to 60, 15 to 40, 15 to 45, 18 to 30, 18 to 40, to 30, 20 to 40, 21 to 25, 21 to 50, 22 to 45, 25 to 40, and any length between the stated ranges. In some embodiments, the primers are usually not more than about 6, 7, 8, 9, 10, 12, 15, 20, 21, 22, 23, 24, 25, 26, 27, 28, 29, 30, 35, 40, 45, 50, 55, 60, 65, or 70 nucleotides in length.

“Incorporating,” as used herein, means becoming part of a nucleic acid polymer.

The term “in the absence of exogenous manipulation” as used herein refers to there being modification of a nucleic acid molecule without changing the solution in which the nucleic acid molecule is being modified. In specific embodiments, it occurs in the absence of the hand of man or in the absence of a machine that changes solution conditions, which may also be referred to as buffer conditions. However, changes in temperature may occur during the modification.

A “nucleoside” is a base-sugar combination, i.e., a nucleotide lacking a phosphate. It is recognized in the art that there is a certain inter-changeability in usage of the terms nucleoside and nucleotide. For example, the nucleotide deoxyuridine triphosphate, dUTP, is a deoxyribonucleoside triphosphate. After incorporation into DNA, it serves as a DNA monomer, formally being deoxyuridylate, i.e., dUMP or deoxyuridine monophosphate. One may say that one incorporates dUTP into DNA even though there is no dUTP moiety in the resultant DNA. Similarly, one may say that one incorporates deoxyuridine into DNA even though that is only a part of the substrate molecule.

“Nucleotide,” as used herein, is a term of art that refers to a base-sugar-phosphate combination. Nucleotides are the monomeric units of nucleic acid polymers, i.e., of DNA and RNA. The term includes ribonucleotide triphosphates, such as rATP, rCTP, rGTP, or rUTP, and deoxyribonucleotide triphosphates, such as dATP, dCTP, dUTP, dGTP, or dTTP.

The term “nucleic acid” or “polynucleotide” will generally refer to at least one molecule or strand of DNA, RNA, DNA-RNA chimera or a derivative or analog thereof, comprising at least one nucleobase, such as, for example, a naturally occurring purine or pyrimidine base found in DNA (e.g., adenine “A,” guanine “G,” thymine “T” and cytosine “C”) or RNA (e.g. A, G, uracil “U” and C). The term “nucleic acid” encompasses the terms “oligonucleotide” and “polynucleotide.” “Oligonucleotide,” as used herein, refers collectively and interchangeably to two terms of art, “oligonucleotide” and “polynucleotide.” Note that although oligonucleotide and polynucleotide are distinct terms of art, there is no exact dividing line between them and they are used interchangeably herein. The term “adapter” may also be used interchangeably with the terms “oligonucleotide” and “polynucleotide.” In addition, the term “adapter” can indicate a linear adapter (either single stranded or double stranded) or a stem-loop adapter. These definitions generally refer to at least one single-stranded molecule, but in specific embodiments will also encompass at least one additional strand that is partially, substantially, or fully complementary to at least one single-stranded molecule. Thus, a nucleic acid may encompass at least one double-stranded molecule or at least one triple-stranded molecule that comprises one or more complementary strand(s) or “complement(s)” of a particular sequence comprising a strand of the molecule. As used herein, a single stranded nucleic acid may be denoted by the prefix “ss,” a double-stranded nucleic acid by the prefix “ds,” and a triple stranded nucleic acid by the prefix “ts.”

A “nucleic acid molecule” refers to any single-stranded or double-stranded nucleic acid molecule including standard canonical bases, hypermodified bases, non-natural bases, or any combination of the bases thereof. For example and without limitation, the nucleic acid molecule contains the four canonical DNA bases—adenine, cytosine, guanine, and thymine, and/or the four canonical RNA bases—adenine, cytosine, guanine, and uracil. Uracil can be substituted for thymine when the nucleoside contains a 2′-deoxyribose group. The nucleic acid molecule can be transformed from RNA into DNA and from DNA into RNA. For example, and without limitation, mRNA can be created into complementary DNA (cDNA) using reverse transcriptase and DNA can be created into RNA using RNA polymerase. A nucleic acid molecule can be of biological or synthetic origin. Examples of nucleic acid molecules include genomic DNA, cDNA, RNA, a DNA/RNA hybrid, amplified DNA, a pre-existing nucleic acid library, etc. A nucleic acid may be obtained from a human sample, such as blood, serum, plasma, cerebrospinal fluid, cheek scrapings, biopsy, semen, urine, feces, saliva, sweat, etc. A nucleic acid molecule may be subjected to various treatments, such as repair treatments and fragmenting treatments. Fragmenting treatments include mechanical, sonic, and hydrodynamic shearing. Repair treatments include nick repair via extension and/or ligation, polishing to create blunt ends, removal of damaged bases, such as deaminated, derivatized, abasic, or crosslinked nucleotides, etc. A nucleic acid molecule of interest may also be subjected to chemical modification (e.g., bisulfite conversion, methylation/demethylation), extension, amplification (e.g., PCR, isothermal, etc.), etc.

Nucleic acid(s) that are “complementary” or “complement(s)” are those that are capable of base-pairing according to the standard Watson-Crick, Hoogsteen or reverse Hoogsteen binding complementarity rules. As used herein, the term “complementary” or “complement(s)” may refer to nucleic acid(s) that are substantially complementary, as may be assessed by the same nucleotide comparison set forth above. The term “substantially complementary” may refer to a nucleic acid comprising at least one sequence of consecutive nucleobases, or semiconsecutive nucleobases if one or more nucleobase moieties are not present in the molecule, are capable of hybridizing to at least one nucleic acid strand or duplex even if less than all nucleobases do not base pair with a counterpart nucleobase. In certain embodiments, a “substantially complementary” nucleic acid contains at least one sequence in which about 70%, about 71%, about 72%, about 73%, about 74%, about 75%, about 76%, about 77%, about 77%, about 78%, about 79%, about 80%, about 81%, about 82%, about 83%, about 84%, about 85%, about 86%, about 87%, about 88%, about 89%, about 90%, about 91%, about 92%, about 93%, about 94%, about 95%, about 96%, about 97%, about 98%, about 99%, to about 100%, and any range therein, of the nucleobase sequence is capable of base-pairing with at least one single or double-stranded nucleic acid molecule during hybridization. In certain embodiments, the term “substantially complementary” refers to at least one nucleic acid that may hybridize to at least one nucleic acid strand or duplex in stringent conditions. In certain embodiments, a “partially complementary” nucleic acid comprises at least one sequence that may hybridize in low stringency conditions to at least one single or double-stranded nucleic acid, or contains at least one sequence in which less than about 70% of the nucleobase sequence is capable of base-pairing with at least one single or double-stranded nucleic acid molecule during hybridization.

The term “non-complementary” refers to nucleic acid sequence that lacks the ability to form at least one Watson-Crick base pair through specific hydrogen bonds.

The term “degenerate” as used herein refers to a nucleotide or series of nucleotides wherein the identity can be selected from a variety of choices of nucleotides, as opposed to a defined sequence. In specific embodiments, there can be a choice from two or more different nucleotides. In further specific embodiments, the selection of a nucleotide at one particular position comprises selection from only purines, only pyrimidines, or from non-pairing purines and pyrimidines.

The term “secondary structure” as used herein refers to the set of interactions between bases pairs. For example, in a DNA double helix, the two strands of DNA are held together by hydrogen bonds. The secondary structure is responsible for the shape that the nucleic acid assumes. For a single stranded nucleic acid, the simplest secondary structure is linear. For a linear secondary structure, no two subsequences of a nucleic acid molecule form an intramolecular structure stronger than −2 kcal/mol. As another example for a single stranded nucleic acid, one portion of the nucleic acid molecule may hybridize with a second portion of the same nucleic acid molecule, thereby forming a hairpin to stem loop secondary structure. For a non-linear secondary structure, at least two subsequences of a nucleic acid molecule from an intramolecular structure stronger than −2 kcal/mol.

As used herein, the term “subsequence” refers to a sequence of at least 5 contiguous base pairs.

As used herein, the term “mutant DNA Template” or “variant DNA Template” refer to the nucleotide sequence of a nucleic acid that harbors a desired allele, such as a single nucleotide polymorphism, to be amplified, identified, or otherwise isolated. As used herein, the term “wildtype sequence” or “background sequence” refers to the nucleotide sequence of a nucleic acid that does not harbor the desired allele. For example, in some instances, the background sequence harbors the wild-type allele whereas the variant sequence harbors the mutant allele. Thus, in some instance, the background sequence and the variant sequence are derived from a common locus in a genome such that the sequences of each may be substantially homologous except for a region harboring the desired allele, nucleotide or group or nucleotides that varies between the two.

“Sample” means a material obtained or isolated from a fresh or preserved biological sample or synthetically created source that contains nucleic acids of interest. Samples can include at least one cell, fetal cell, cell culture, tissue specimen, blood, serum, plasma, saliva, urine, tear, vaginal secretion, sweat, lymph fluid, cerebrospinal fluid, mucosa secretion, peritoneal fluid, ascites fluid, fecal matter, body exudates, umbilical cord blood, chorionic villi, amniotic fluid, embryonic tissue, multicellular embryo, lysate, extract, solution, or reaction mixture suspected of containing immune nucleic acids of interest. Samples can also include non-human sources, such as non-human primates, rodents and other mammals, other animals, plants, fungi, bacteria, and viruses.

As used herein in relation to a nucleotide sequence, “substantially known” refers to having sufficient sequence information in order to permit preparation of a nucleic acid molecule, including its amplification. This will typically be about 100%, although in some embodiments some portion of an adapter sequence is random or degenerate. Thus, in specific embodiments, substantially known refers to about 50% to about 100%, about 60% to about 100%, about 70% to about 100%, about 80% to about 100%, about 90% to about 100%, about 95% to about 100%, about 97% to about 100%, about 98% to about 100%, or about 99% to about 100%.

As used herein, “essentially free,” in terms of a specified component, is used herein to mean that none of the specified component has been purposefully formulated into a composition and/or is present only as a contaminant or in trace amounts. The total amount of the specified component resulting from any unintended contamination of a composition is therefore well below 0.05%, preferably below 0.01%. Most preferred is a composition in which no amount of the specified component can be detected with standard analytical methods.

As used herein the specification, “a” or “an” may mean one or more. As used herein in the claim(s), when used in conjunction with the word “comprising,” the words “a” or “an” may mean one or more than one.

The use of the term “or” in the claims is used to mean “and/or” unless explicitly indicated to refer to alternatives only or the alternatives are mutually exclusive, although the disclosure supports a definition that refers to only alternatives and “and/or.” As used herein “another” may mean at least a second or more.

Throughout this application, the term “about” is used to indicate that a value includes the inherent variation of error for the device, the method being employed to determine the value, the variation that exists among the study subjects, or a value that is within 10% of a stated value.

### II. ACE in Quantitative PCR

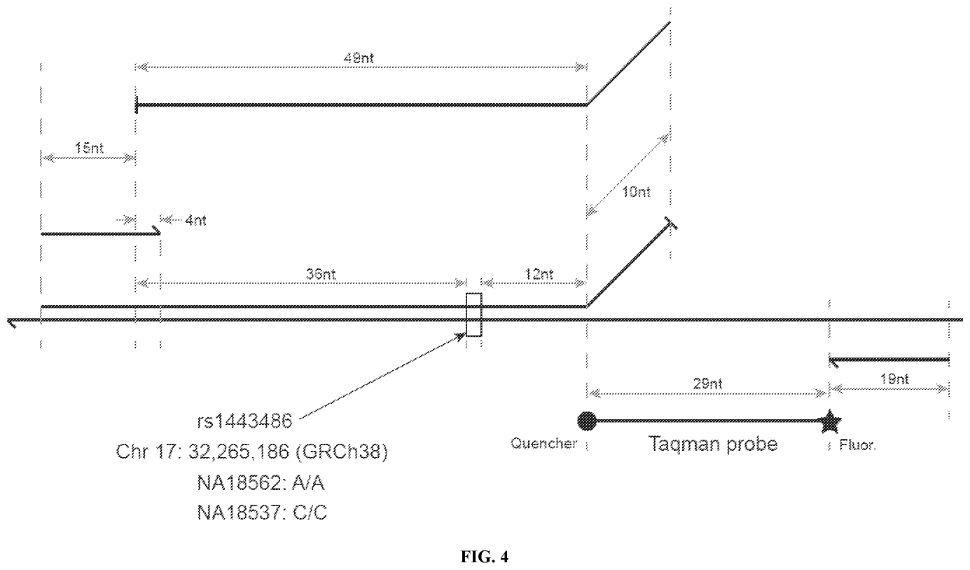

ACE may be used in a qPCR setting. In this embodiment, a Forward Primer oligonucleotide, a Reverse Primer oligonucleotide, a Suppressor oligonucleotide, an Auxiliary oligonucleotide, a DNA polymerase, dNTPs, and buffers needed for PCR are mixed with a Sample possibly comprising wildtype DNA Template molecules and possibly comprising mutant DNA Template molecules. In some embodiments, the mixture further comprises a Taqman probe. FIG. 4 illustrates a specific embodiment of an ACE system for enriching alleles other than the A allele at single nucleotide polymorphism (SNP) locus rs1443486. The NA18562 human genomic DNA is homozygous for the A allele, and the NA18537 human genomic DNA is homozygous for the C allele on the (−) strand of DNA. A Taqman probe that bind downstream of the Suppressor oligonucleotide may be included to produce a specific fluorescence signal for the amplicons generated. In this example, the Suppressor oligonucleotide is designed to perfectly match the NA18562 A allele.

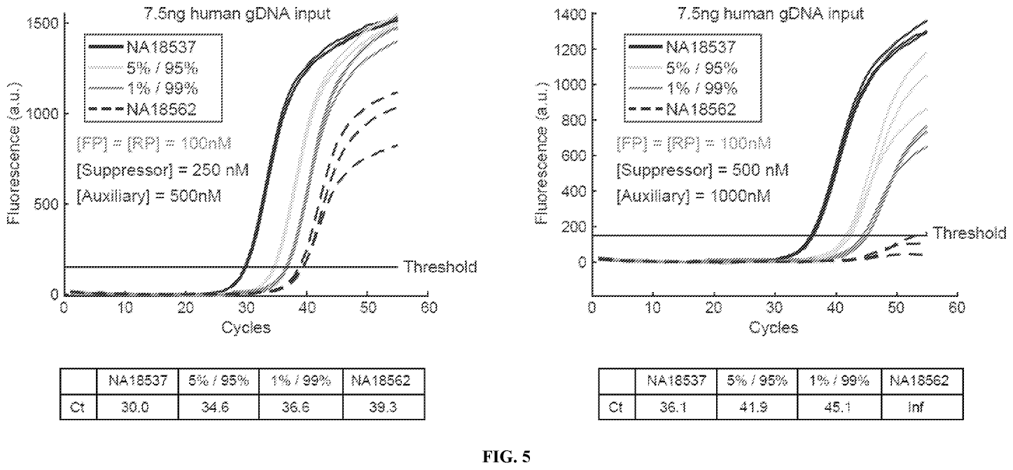

This ACE-qPCR set up was used to enrich and detect non-A alleles at rs1443486. Because the NA18562 human genomic DNA is homozygous for the A allele, it is considered the wildtype Template molecule for this reaction. The NA18537 human genomic DNA is homozygous for the C allele and is the mutant Template molecule. The qPCR reaction was able to clearly distinguish between 100% NA18537, 5% NA18537/95% NA18562, 1% NA18537/99% NA18562, and 100% NA18562 (FIG. 5). Even 1% NA18537 in 99% NA18562 can be clearly distinguished from 100% NA18562, implying over 100-fold enrichment of the C allele over the A allele. Higher concentrations of Suppressor oligonucleotide led to delayed Ct values for all DNA samples. The stoichiometric (i.e., molar) ratio of the Auxiliary oligonucleotide to the Suppressor oligonucleotide or of the Forward Primer oligonucleotide to the Suppressor oligonucleotide may be between 0.8 and 100, 0.9 and 100, 1 and 100, 2 and 100, 3 and 100, 4 and 100, 5 and 100, 10 and 100, 15 and 100, 20 and 100, 25 and 100, 30 and 100, 40 and 100, 50 and 100, 0.8 and 50, 0.8 and 45, 0.8 and 40, 0.8 and 35, 0.8 and 30, 0.8 and 25, 0.8 and 20, 1 and 50, 1 and 45, 1 and 40, 1 and 35, 1 and 30, 1 and 25, or 1 and 20. The stoichiometric (i.e., molar) ratio of the Auxiliary oligonucleotide to the Suppressor oligonucleotide or of the Forward Primer oligonucleotide to the Suppressor oligonucleotide may be at least or about 0.8, 0.9, 1, 1.1, 1.2, 1.3, 1.4, 1.5, 2, 3, 4, 5, 6, 7, 8, 9, 10, 15, 20, 25, 30, 35, 40, 45, 50, 55, 60, 65, 70, 75, 80, 85, 90, 95, or 100.

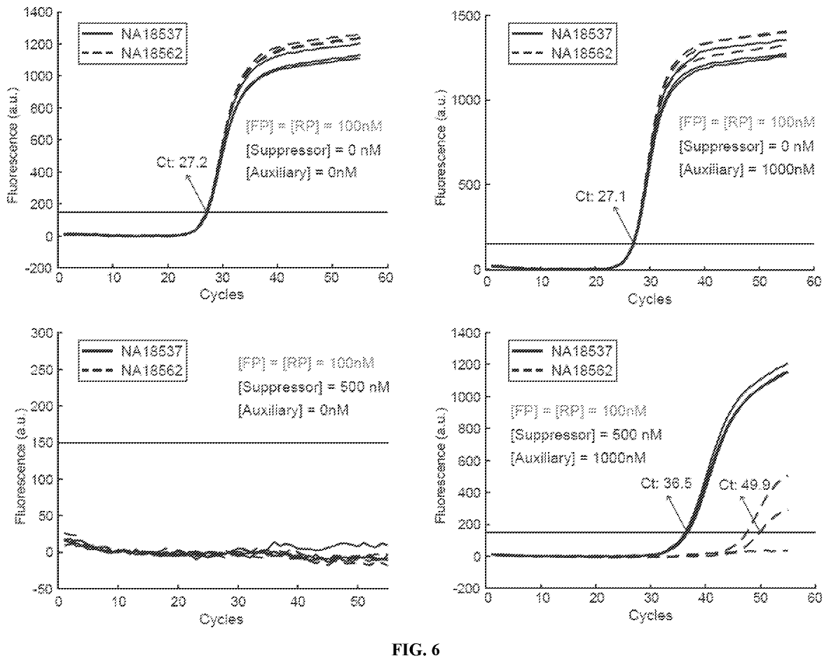

Enrichment of NA18537's C allele over NA18562's A allele is not achieved when either the Suppressor oligonucleotide or the Auxiliary oligonucleotide is absent (FIG. 6). When both are absent, the cycle threshold (Ct) values are similar, suggesting that the input DNA quantities are similar and the PCR amplification efficiencies are similar. When only Auxiliary oligonucleotide is present, the Ct values for both NA18537 and NA18562 are unchanged, suggesting that the binding of Auxiliary oligonucleotide to the reverse complement of the Template molecule does not inhibit the PCR reaction. When only the Suppressor oligonucleotide is present, no amplification is observed, suggesting that in the absence of the Auxiliary oligonucleotide, the Suppressor oligonucleotide binding to the Template molecule is irreversible and both mutant Template molecules and wildtype Template molecules are unable to amplify. Only when both Suppressor oligonucleotide and Auxiliary oligonucleotide are present is there differential amplification of mutant and wildtype Template molecules. In other words, when both Suppressor oligonucleotide and Auxiliary oligonucleotide are present, then preferential amplification of NA18537 is observed, because its Template molecule is mismatched against the Suppressor oligonucleotide.

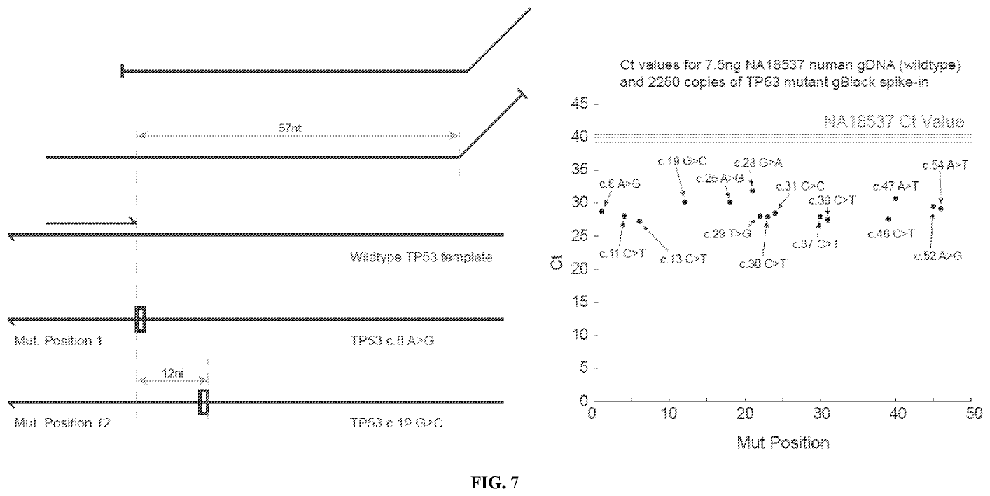

To demonstrate that ACE enriches all Template molecules with sequence variations within the Target Subsequence, an ACE system was designed to target the human TP53 gene (FIG. 7). This ACE system was tested using qPCR using 15 separate TP53 mutations at different loci spanning across the 50 nt Target Subsequence. This experiment was performed using synthetic gBlock oligonucleotide templates (606 nt long each) as mutant Template molecules. The observed Ct values of 7.5 ng NA18537 without mutant Template molecules were plotted as a line and have values of about 40. The Ct values of the mutant Template samples were all roughly 9 to 13 cycles lower, at between 27 and 31 (FIG. 7). Based on the design of the ACE mechanism, all mutations were selectively enriched by the same ACE Suppressor oligonucleotide and Auxiliary oligonucleotide regardless of the mutation's position on the Suppressor oligonucleotide.

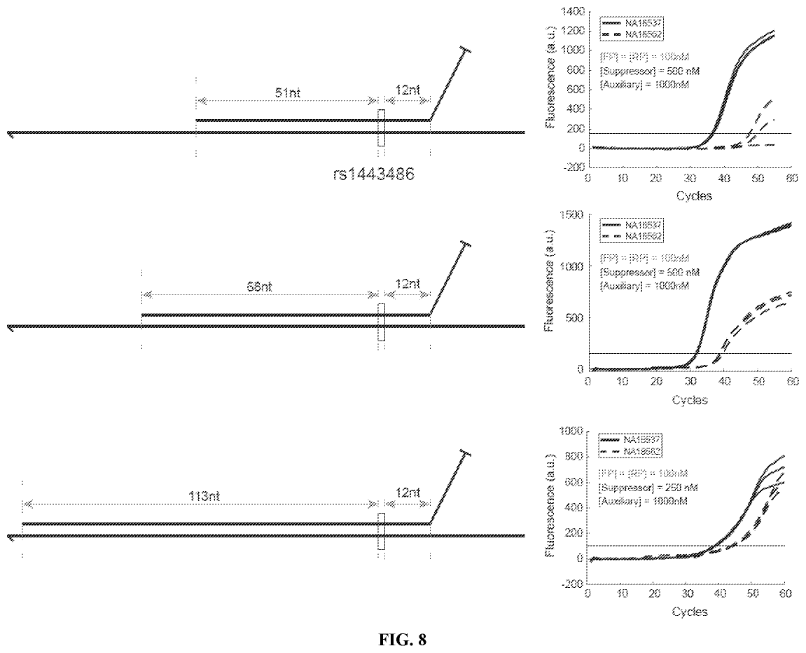

Finally, to demonstrate that the length of the Target Subsequence can be extended through rational design of the Suppressor oligonucleotide and the Auxiliary oligonucleotide, three separate ACE sets targeting the same human SNP (rs1443486) with different Target Subsequence lengths of 64 nt (51+1+12), 81 nt, and 126 nt were constructed (FIG. 8). The SNP position was designed to be consistently the 13th nucleotide from the end of the Template-binding region. All three ACE systems showed significant Ct value differences between the NA18537 variant Template and the NA18562 wildtype Template. Significant delay was observed for the longest Suppressor oligonucleotide, suggesting either that that longer length necessitates a longer anneal cycle time to allow strand displacement, or that the longer length causes the Auxiliary oligonucleotide purity to drop due to the lower purities of longer chemically synthesized oligonucleotides, rendering displacement less efficient.

### III. ACE in Next Generation Sequencing (NGS) Library Preparation

In some embodiments, ACE can be used for variant enrichment during the library preparation process of a high-throughput sequencing procedure. In some embodiments, the high-throughput sequencing is a sequencing-by-synthesis (NGS) method. In other embodiments, the high-throughput sequencing is performed via electrical current measurements in conjunction with a nanopore.

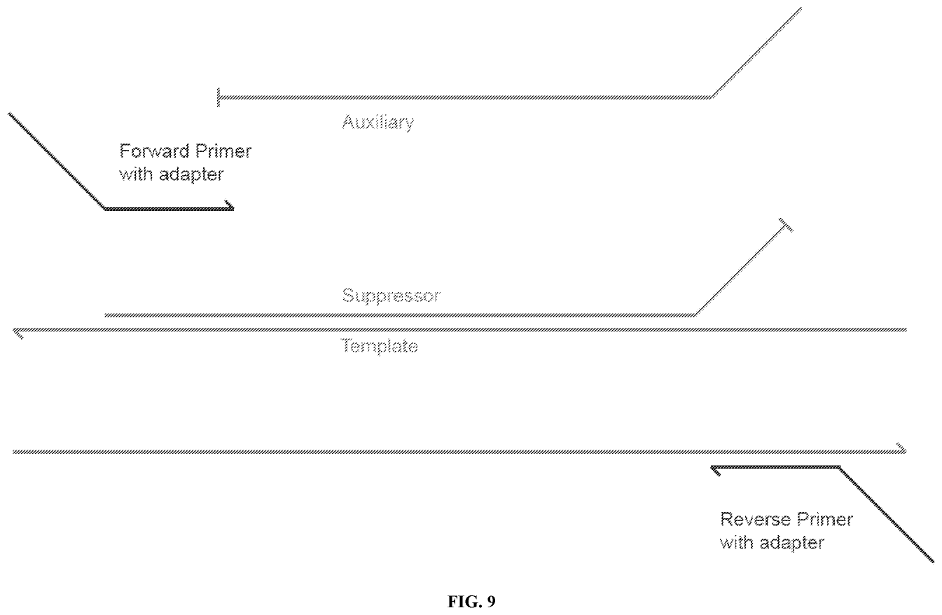

Multiple ACE systems can be designed to enrich variants in different genetic regions of interest in a library. In some embodiments, the ACE systems can be applied in multiplex PCR using standard-length gene specific primers, followed by adapter PCR or adapter ligation to append sequencing-specific adapter sequences. Methods of using adaptor ligation to add additional sequences are described, e.g., in U.S. Pat. No. 7,803,550, which is incorporated by reference herein in its entirety. In other embodiments, the ACE systems can utilize Forward Primer oligonucleotides and Reverse Primer oligonucleotides with adapter sequences at the 5′ ends as part of the ACE enrichment (FIG. 9). The adapter sequences allow subsequent adapter PCR for next-generation sequencing (NGS) library preparation. In various embodiments, a multiplex ACE panel can comprise 2, 3, 4, 5, 6, 7, 8, 9, 10, 11, 12, 13, 14, 15, 16, 17, 18, 19, 20, 25, 30, 35, 40, 45, 50, 55, 60, 65, 70, 75, 80, 85, 90, 95, or 100 ACE systems.

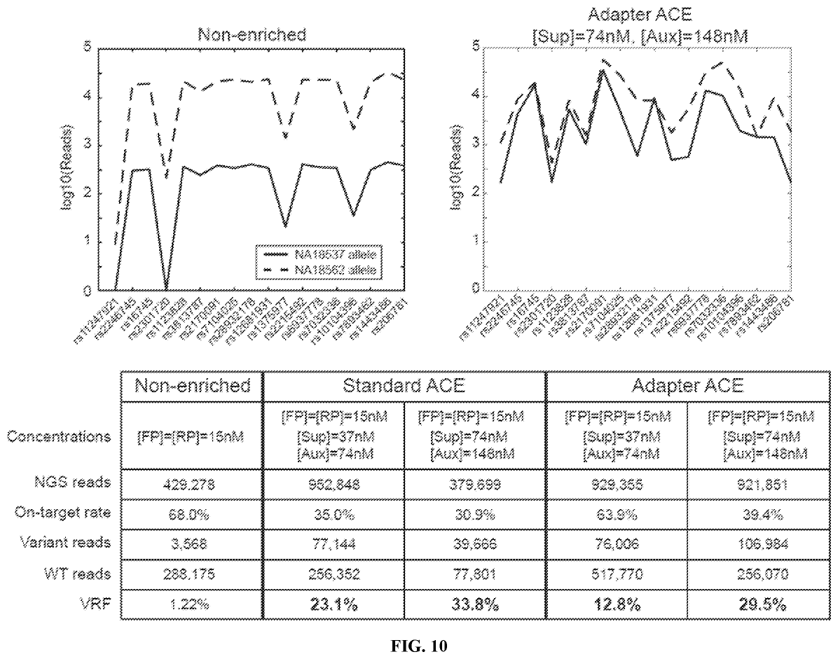

As an example, an 18-plex ACE panel, targeting 18 different SNP loci in which NA18537 and NA18562 were homozygous for different alleles, was constructed. This 18-plex ACE panel was designed to suppress the homozygous SNP alleles of the NA18562 sample. All 18 Suppressor oligonucleotides were designed to be perfectly matched against the NA18562 alleles. The 18-plex ACE was tested on a sample of 1% NA18537/99% NA18562; each library used 25 ng of this mixture as input. An Illumina MiSeq was used for performing NGS. Without the ACE Suppressor oligonucleotide and Auxiliary oligonucleotide, the number of NGS reads mapping to the NA18562 allele was roughly 100-fold higher than the number of NGS reads mapping to the NA18537 allele at every locus, as expected (FIG. 10). In the libraries with the ACE Suppressor oligonucleotide and Auxiliary oligonucleotide present, the relative fraction of reads mapping to the NA18537 variant allele was significantly increased for all loci. Overall, the fraction of NGS reads mapped to the NA18537 loci was increased from 1.22% to up to 33.8%, a weighted average enrichment of more than 24-fold. Based on these single-plex qPCR ACE results, the ACE fold-enrichment can be significantly further improved through the optimization of sequences, concentrations, reaction times, and other experimental protocol minutiae.

### IV. Tiled ACE

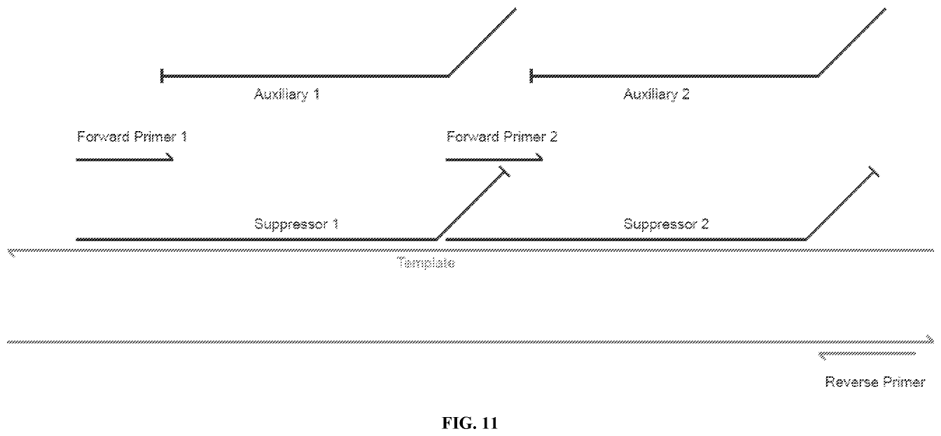

In some embodiments, the ACE method can be used with multiple Suppressor oligonucleotides that tile or mostly tile a continuous DNA region. This would circumvent challenges in synthesizing high purity long DNA oligonucleotides for long Suppressor oligonucleotides. FIG. 11 shows an embodiment of ACE in PCR in which two different Suppressor oligonucleotides are paired with different corresponding Auxiliary oligonucleotide and Forward Primer oligonucleotides that bind adjacent sequences on the Template molecules. In various embodiments, 2, 3, 4, 5, 6, 7, 8, 9, 10, or more Suppressor oligonucleotides, each paired with its own Auxiliary oligonucleotides, may be used to tile or mostly tile a continuous DNA region.

### V. Gene Fusion Enrichment with PCR

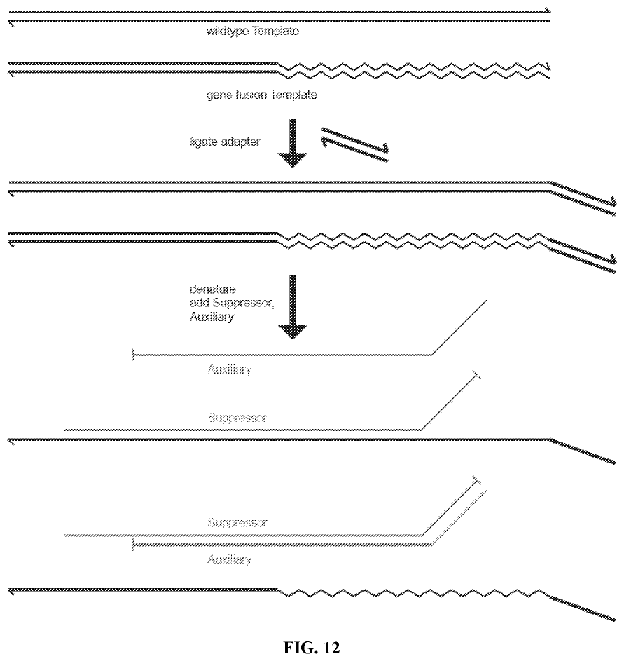

In some embodiments, the ACE method can be used to enrich gene fusions in which a part of a gene of interest has been rearranged to be next to part of another gene, such as through a chromosomal translocation. In this situation, the downstream or upstream gene fusion partner may not be a priori known. FIG. 12 shows an embodiment in which the DNA Template molecule potentially containing a gene fusion is first ligated to an adapter. Methods of using adaptor ligation to add additional sequences are described, e.g., in U.S. Pat. No. 7,803,550, which is incorporated by reference herein in its entirety. ACE-PCR is performed using a Reverse Primer oligonucleotide that is reverse complementary to the adapter sequence. Note that the adapter for this figure and embodiment is not necessarily a sequencing adapter and can in principle be any designed DNA sequence.

### VI. ACE in Rolling Circle Amplification (RCA)

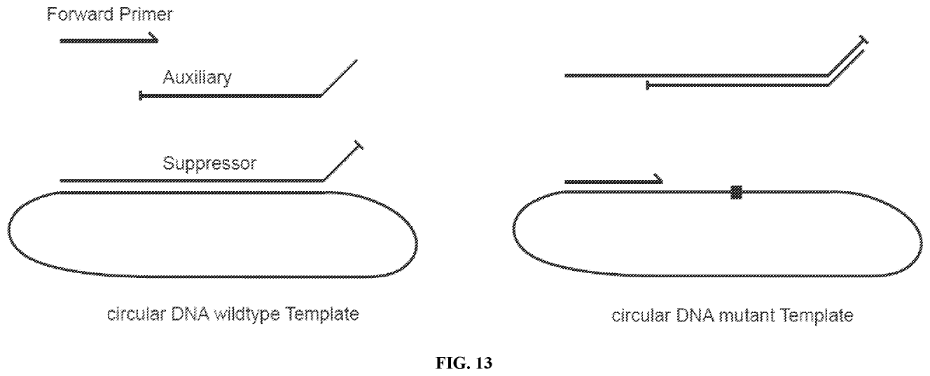

In some embodiments, the ACE method does not require a reverse primer. FIG. 13 shows an embodiment in which ACE is applied in RCA. The circular DNA Template molecule can be circular biological DNA sequences, or it can be constructed from linear DNA sequences circularized through an enzymatic method. The ACE Suppressor oligonucleotide prevents Forward Primer oligonucleotide binding, and by extension prevents RCA on wildtype Template molecules.

### VII. Fold-Enrichment Analysis and VAF Quantitation

The fold-enrichment (EF) for a variant Template molecule is defined as the relative amplification of the variant Template molecule over the corresponding wildtype Template molecule. In general, larger number of PCR cycles with ACE result in larger EF values. In an NGS library setting, the values of VRF, EF, and variant allele frequency (VAF) satisfy the following equations:

VRF=(VAF*EF)/(VAF*EF+(1−VAF))

VAF=(VRF)/(VRF*(1−EF)+EF)

EF=(VRF*(VAF−1))/(VAF*(VRF−1))

Given the known values of any two of the three variables, the last variable can be calculated. Thus, during initial calibration experiments, VRF and VAF from known samples can be used to calculate EF. Afterwards, when running NGS on samples with unknown VAFs, VRF and EF can be used to calculate the value of VAF.

### VIII. UMI Designs

The concept of UMI is to give every original DNA molecule a different DNA sequence as a “barcode,” so that the origin of each NGS read can be tracked based on the barcode sequence. Given enough NGS reads, the number of unique UMIs found in the NGS output can reflect the number of original DNA molecules. Labeling each original molecule uniquely is achieved by using a large number of different UMI sequences; for example, using 109 different UMI sequences for 100,000 original molecules will generate <0.006% molecules carrying repeated UMIs.

DNA sequences containing degenerate bases, such as poly(N) (i.e., a mix of A, T, C, or G at each position), are often used as UMI sequences. In QBDA, poly(H) (i.e., as mix of A, T, or C at each position) is used as the UMI because it has weaker cross-binding energy compared to poly(N) or a mix of S (C or G) and W (A or T) bases. (H)₂₀ contains 3.5×109 different sequences, which are enough for 100,000 molecules as input; (H)₁₅ contains 1.4×10⁷ different sequences, which are enough for 6,000 molecules as input.

A specific DNA-based barcode that serves as a method of error correction has been developed. Like any assay, NGS may produce misreads. This DNA-based barcode, a 7 nucleotide Hamming barcode, allows for the identification of misreads and correction of these errors.

Naive design of barcode sequences can result in barcodes sequence that are susceptible to NGS intrinsic error. In the field of signal processing, passing messages across faulty channels (e.g., the Internet) has led to the development of error correcting and error detecting codes. These ideas can be directly applied in barcode design. Because Illumina sequencing errors are predominantly base replacements (as opposed to insertions or deletions), Hamming encoding is well-suited for barcode sequences.

To review, the simplest (7,4) Hamming code inserts 3 error-correcting bits for every 4-bit message (longer messages are first broken up into 4-bit words). All 7-bit instances of the Hamming code have the property that they are at least Hamming distance 3 from any other instance—that is to say, one would need to change at least 3 bits in order to transform one Hamming code instance into another. This property means that (7,4) Hamming codes are correcting for up to one error, and tolerant for up to two errors: The original sequence can be restored from any sequence mutated by one base; more conservatively, any sequence with two mutations will not match any other code and can be excluded.

For example, (7,4) DNA barcodes can be used. The assignment of A, T, C, and G to numerical values and the design of the error check equations are selected such that long homopolymers and extreme G/C content are rare. Manual pruning of the 256 possible (7,4) Hamming codes removes 40 sequences that can contribute to homopolymers of more than 5 nt (via having a homopolymer of length 3 at the beginning or end of the barcode) or have G/C content of >75% or <25%, resulting in 216 good (7,4) nt barcode segments.

For demonstration purposes, 21 nt barcodes, corresponding to three (7,4) barcode segments, which can enumerate over 10 million distinct barcodes, were used. These barcodes can correct 1 nt error every 7 nt, or tolerate 2 nt errors every 7 nt. At a 1% intrinsic error rate, the proposed barcodes exhibit roughly 0.6% error rate when NGS reads unmatched to any designed barcodes are corrected, and 0.01% error when unmatched NGS reads are discarded. These are roughly 20-fold and 1000-fold better than a naive barcode with no error correction. The probability of having 3 or more errors in a block of 7 nt is 0.0034%, and unlikely to significantly affect the interpretation of SNP genotypes due to position-specific primer bleeding.

Correction of NGS reads that do not match any designed barcodes is done at the level of satisfying error-checking equations, and does not require knowledge of the designed barcode sequences. The time complexity of this operation is O(M), where M is the length of the barcode (here M=21). After correcting or discarding NGS reads that do not exactly match any designed barcode, a Suffix Tree algorithm can be used to perform exact string matching on the designed barcodes. Suffix Tree is extremely rapid, with runtime complexity of O(M); importantly it has no dependence on the number of barcodes designed, and thus scales well to be highly multiplex.

### IX. Thermodynamic Calculations

Methods for the calculation of ΔG° values from sequences are known in the art. There exist different conventions for calculating the ΔG° of different region interactions. WO2015/179339, which is incorporated herein by reference in its entirety, provides exemplary energy calculations based on the nearest neighbor model. The calculation of ΔG°₁, ΔG°₂, ΔG°₃, and ΔG°₄ from the primer sequence, Suppressor oligonucleotide sequence, Auxiliary oligonucleotide sequence, Target molecule sequence, variant sequence, operational temperature, and operational buffer conditions are known to those skilled in the art. The operational temperature may be about 20° C., about 25° C., about 30° C., about 35° C., about 40° C., about 45° C., about 50° C., about 55° C., about 60° C., about 65° C., or about 70° C. The operational buffer conditions may be buffer conditions suitable for PCR.

### X. Further Processing of Target Nucleic Acids

A. Amplification of DNA

A number of template-dependent processes are available to amplify the nucleic acids present in a given template sample. One of the best known amplification methods is the polymerase chain reaction (referred to as PCR′) which is described in detail in U.S. Pat. Nos. 4,683,195, 4,683,202, and 4,800,159 and in Innis et al., 1990, each of which is incorporated herein by reference in their entirety. Briefly, two synthetic oligonucleotide primers, which are complementary to two regions of the template DNA (one for each strand) to be amplified, are added to the template DNA (that need not be pure), in the presence of excess deoxynucleotides (dNTP's) and a thermostable polymerase, such as, for example, Taq (Thermus aquaticus) DNA polymerase. In a series (typically 30-35) of temperature cycles, the target DNA is repeatedly denatured (around 90° C.), annealed to the primers (typically at 50-60° C.) and a daughter strand extended from the primers (72° C.). As the daughter strands are created they act as templates in subsequent cycles. Thus, the template region between the two primers is amplified exponentially, rather than linearly.

B. Sequencing of DNA

Methods are also provided for the sequencing of the library of adaptor-linked fragments. Any technique for sequencing nucleic acids known to those skilled in the art can be used in the methods of the present disclosure. DNA sequencing techniques include classic dideoxy sequencing reactions (Sanger method) using labeled terminators or primers and gel separation in slab or capillary, sequencing-by-synthesis using reversibly terminated labeled nucleotides, pyrosequencing, 454 sequencing, allele specific hybridization to a library of labeled oligonucleotide probes, sequencing-by-synthesis using allele specific hybridization to a library of labeled clones that is followed by ligation, real time monitoring of the incorporation of labeled nucleotides during a polymerization step, and SOLiD sequencing.

The nucleic acid library may be generated with an approach compatible with Illumina sequencing such as a Nextera™ DNA sample prep kit, and additional approaches for generating Illumina next-generation sequencing library preparation are described, e.g., in Oyola et al. (2012). In other embodiments, a nucleic acid library is generated with a method compatible with a SOLiD™ or Ion Torrent sequencing method (e.g., a SOLiD® Fragment Library Construction Kit, a SOLiD® Mate-Paired Library Construction Kit, SOLiD® ChIP-Seq Kit, a SOLiD® Total RNA-Seq Kit, a SOLiD® SAGE™ Kit, a Ambion® RNA-Seq Library Construction Kit, etc.). Additional methods for next-generation sequencing methods, including various methods for library construction that may be used with embodiments of the present invention are described, e.g., in Pareek (2011) and Thudi (2012).

In particular aspects, the sequencing technologies used in the methods of the present disclosure include the HiSeq™ system (e.g., HiSeq™ 2000 and HiSeq™ 1000), the NextSeq™ 500, and the MiSeq™ system from Illumina, Inc. The HiSeq™ system is based on massively parallel sequencing of millions of fragments using attachment of randomly fragmented genomic DNA to a planar, optically transparent surface and solid phase amplification to create a high density sequencing flow cell with millions of clusters, each containing about 1,000 copies of template per sq. cm. These templates are sequenced using four-color DNA sequencing-by-synthesis technology. The MiSeq™ system uses TruSeq™, Illumina's reversible terminator-based sequencing-by-synthesis.

Another example of a DNA sequencing technique that can be used in the methods of the present disclosure is 454 sequencing (Roche) (Margulies et al., 2005). 454 sequencing involves two steps. In the first step, DNA is sheared into fragments of approximately 300-800 base pairs, and the fragments are blunt ended. Oligonucleotide adaptors are then ligated to the ends of the fragments. The adaptors serve as primers for amplification and sequencing of the fragments. The fragments can be attached to DNA capture beads, e.g., streptavidin-coated beads using, e.g., Adaptor B, which contains 5′-biotin tag. The fragments attached to the beads are PCR amplified within droplets of an oil-water emulsion. The result is multiple copies of clonally amplified DNA fragments on each bead. In the second step, the beads are captured in wells (pico-liter sized). Pyrosequencing is performed on each DNA fragment in parallel. Addition of one or more nucleotides generates a light signal that is recorded by a CCD camera in a sequencing instrument. The signal strength is proportional to the number of nucleotides incorporated.

Another example of a DNA sequencing technique that can be used in the methods of the present disclosure is SOLiD technology (Life Technologies, Inc.). In SOLiD sequencing, genomic DNA is sheared into fragments, and adaptors are attached to the 5′ and 3′ ends of the fragments to generate a fragment library. Alternatively, internal adaptors can be introduced by ligating adaptors to the 5′ and 3′ ends of the fragments, circularizing the fragments, digesting the circularized fragment to generate an internal adaptor, and attaching adaptors to the 5′ and 3′ ends of the resulting fragments to generate a mate-paired library. Next, clonal bead populations are prepared in microreactors containing beads, primers, template, and PCR components. Following PCR, the templates are denatured and beads are enriched to separate the beads with extended templates. Templates on the selected beads are subjected to a 3′ modification that permits bonding to a glass slide.

Another example of a DNA sequencing technique that can be used in the methods of the present disclosure is the Ion Torrent system (Life Technologies, Inc.). Ion Torrent uses a high-density array of micro-machined wells to perform this biochemical process in a massively parallel way. Each well holds a different DNA template. Beneath the wells is an ion-sensitive layer and beneath that a proprietary Ion sensor. If a nucleotide, for example a C, is added to a DNA template and is then incorporated into a strand of DNA, a hydrogen ion will be released. The charge from that ion will change the pH of the solution, which can be detected by the proprietary ion sensor. The sequencer will call the base, going directly from chemical information to digital information. The Ion Personal Genome Machine (PGM™) sequencer then sequentially floods the chip with one nucleotide after another. If the next nucleotide that floods the chip is not a match, no voltage change will be recorded and no base will be called. If there are two identical bases on the DNA strand, the voltage will be double, and the chip will record two identical bases called. Because this is direct detection—no scanning, no cameras, no light—each nucleotide incorporation is recorded in seconds.

Another example of a sequencing technology that can be used in the methods of the present disclosure includes the single molecule, real-time (SMRT™) technology of Pacific Biosciences. In SMRT™, each of the four DNA bases is attached to one of four different fluorescent dyes. These dyes are phospholinked. A single DNA polymerase is immobilized with a single molecule of template single stranded DNA at the bottom of a zero-mode waveguide (ZMW). A ZMW is a confinement structure which enables observation of incorporation of a single nucleotide by DNA polymerase against the background of fluorescent nucleotides that rapidly diffuse in and out of the ZMW (in microseconds). It takes several milliseconds to incorporate a nucleotide into a growing strand. During this time, the fluorescent label is excited and produces a fluorescent signal, and the fluorescent tag is cleaved off. Detection of the corresponding fluorescence of the dye indicates which base was incorporated. The process is repeated.

A further sequencing platform includes the CGA Platform (Complete Genomics). The CGA technology is based on preparation of circular DNA libraries and rolling circle amplification (RCA) to generate DNA nanoballs that are arrayed on a solid support (Drmanac et al. 2009). Complete genomics' CGA Platform uses a novel strategy called combinatorial probe anchor ligation (cPAL) for sequencing. The process begins by hybridization between an anchor molecule and one of the unique adapters. Four degenerate 9-mer oligonucleotides are labeled with specific fluorophores that correspond to a specific nucleotide (A, C, G, or T) in the first position of the probe. Sequence determination occurs in a reaction where the correct matching probe is hybridized to a template and ligated to the anchor using T4 DNA ligase. After imaging of the ligated products, the ligated anchor-probe molecules are denatured. The process of hybridization, ligation, imaging, and denaturing is repeated five times using new sets of fluorescently labeled 9-mer probes that contain known bases at the n+1, n+2, n+3, and n+4 positions.

A further sequencing platform includes nanopore sequencing (Oxford Nanopore). Nanopore detection arrays are described in US2011/0177498; US2011/0229877; US2012/0133354; WO2012/042226; WO2012/107778, and have been used for nucleic acid sequencing as described in US2012/0058468; US2012/0064599; US2012/0322679 and WO2012/164270, all of which are hereby incorporated by reference. A single molecule of DNA can be sequenced directly using a nanopore, without the need for an intervening PCR amplification step or a chemical labelling step or the need for optical instrumentation to identify the chemical label. Commercially available nanopore nucleic acid sequencing units are developed by Oxford Nanopore (Oxford, United Kingdom). The GridION™ system and miniaturised MinION™ device are designed to provide novel qualities in molecular sensing such as real-time data streaming, improved simplicity, efficiency and scalability of workflows and direct analysis of the molecule of interest. Using the Oxford Nanopore nanopore sequencing platform, an ionic current is passed through the nanopore by setting a voltage across this membrane. If an analyte passes through the pore or near its aperture, this event creates a characteristic disruption in current. Measurement of that current makes it possible to identify the molecule in question. For example, this system can be used to distinguish between the four standard DNA bases G, A, T and C, and also modified bases. It can be used to identify target proteins, small molecules, or to gain rich molecular information, for example to distinguish between the enantiomers of ibuprofen or study molecular binding dynamics. These nanopore arrays are useful for scientific applications specific for each analyte type; for example, when sequencing DNA, the technology may be used for resequencing, de novo sequencing, and epigenetics.

### XI. Kits

The technology described herein includes kits comprising Suppressor oligonucleotides, Auxiliary oligonucleotides, and primers as disclosed herein. Exemplary kits include qPCR kits, Sanger kits, NGS panels, and nanopore sequencing panels. Such panels may provide the necessary reagents for detecting mutations in tumor suppressor genes, such as, for example, TP53, PTEN, BRCA1, and/or BRCA2, with high sensitivity. A “kit” refers to a combination of physical elements. For example, a kit may include, for example, one or more components such as nucleic acid primers, Suppressor oligonucleotides, Auxiliary oligonucleotides, enzymes, reaction buffers, an instruction sheet, and other elements useful to practice the technology described herein. These physical elements can be arranged in any way suitable for carrying out the invention.

The components of the kits may be packaged either in aqueous media or in lyophilized form. The container means of the kits will generally include at least one vial, test tube, flask, bottle, syringe or other container means, into which a component may be placed, and preferably, suitably aliquoted (e.g., aliquoted into the wells of a microtiter plate). Where there is more than one component in the kit, the kit also will generally contain a second, third or other additional container into which the additional components may be separately placed. However, various combinations of components may be comprised in a single vial. The kits of the present invention also will typically include a means for containing the nucleic acids, and any other reagent containers in close confinement for commercial sale. Such containers may include injection or blow molded plastic containers into which the desired vials are retained. A kit will also include instructions for employing the kit components as well the use of any other reagent not included in the kit. Instructions may include variations that can be implemented.

### XII. Examples

The following examples are included to demonstrate preferred embodiments of the invention. It should be appreciated by those of skill in the art that the techniques disclosed in the examples which follow represent techniques discovered by the inventor to function well in the practice of the invention, and thus can be considered to constitute preferred modes for its practice. However, those of skill in the art should, in light of the present disclosure, appreciate that many changes can be made in the specific embodiments which are disclosed and still obtain a like or similar result without departing from the spirit and scope of the invention.

### Example 1—Quantitative PCR (qPCR) Reaction Protocols and Conditions

The final concentrations of the Forward Primer oligonucleotide and Reverse Primer oligonucleotide were typically 100 nM in 15 μL of reaction mixture; however, variations from this are noted in the Figures. The final concentration of the Suppressor oligonucleotide varied between 250 nM and 500 nM and is noted in the Figures. The final concentration of the Auxiliary oligonucleotide varied between 500 nM and 1000 nM and is noted in the Figures. The final concentration of the Taqman probe is typically 50 nM. The PowerUP SybrGreen DNA Polymerase MasterMix (Thermo Fisher) was used for all qPCR experiments. Thermal cycling and fluorescence measurements were performed using a Bio-Rad CFX96 qPCR instrument. The thermal cycling protocol was as follows:

1. 95° C. 3 minutes;

2. 60 cycles of (95° C. for 15 seconds, 60° C. for 90 seconds)

### Example 2—ACE in Quantitative PCR

As an example, an ACE system was designed for enriching alleles other than the A allele at single nucleotide polymorphism (SNP) locus rs1443486. The NA18562 human genomic DNA is homozygous for the A allele, and the NA18537 human genomic DNA is homozygous for the C allele on the (−) strand of DNA. The Suppressor oligonucleotide was designed to perfectly match the NA18562 A allele and had the sequence: ttcctgcagggaaacagcatcgattgttttctttaaaagatcccctactccTttttggctaactGAACCCTGACTT/3 SpC3/(SEQ ID NO: 35; FIG. 4). The Auxiliary oligonucleotide had the sequence: GTCAGGGTTC agttagccaaaaAggagtaggggatcttttaaagaaaacaatcgatgct/3SpC3/(SEQ ID NO: 36). Because the NA18562 human genomic DNA is homozygous for the A allele, it was considered the wildtype Template for this reaction. Since the NA18537 human genomic DNA is homozygous for the C allele, it was considered the mutant Template for this reaction. A Taqman probe (/5Cy5/ggtaaagaaactaaagcaatcagaaagga/3IAbRQSp/; SEQ ID NO: 37) that bind downstream of the Suppressor oligonucleotide was used to produce a specific fluorescence signal for the amplicons generated.

After applying ACE-qPCR to the enrichment and detection of non-A alleles at rs1443486, the cycle threshold (Ct) value of the qPCR reaction can be clearly distinguished between 100% NA18537, 5% NA18537/95% NA18562, 1% NA18537/99% NA18562, and 100% NA18562 (FIG. 5). Even 1% NA18537 in 99% NA18562 can be clearly distinguished from 100% NA18562, implying over 100-fold enrichment of the C allele over the A allele. Higher concentrations of Suppressor oligonucleotide led to delayed Ct values for all DNA samples (FIG. 5).

Enrichment of NA18537's C allele over NA18562's A allele was not achieved when either the Suppressor oligonucleotide or the Auxiliary oligonucleotide was absent (FIG. 6). When both were absent, the cycle threshold (Ct) values were similar, suggesting that the input DNA quantities were similar and the PCR amplification efficiencies were similar. When only Auxiliary oligonucleotide was present, the Ct values for both NA18537 and NA18562 were unchanged, suggesting that the binding of Auxiliary oligonucleotide to the reverse complement of the Template molecule did not inhibit the PCR reaction. When only the Suppressor oligonucleotide was present, no amplification of either Template molecule was observed, suggesting that in the absence of the Auxiliary oligonucleotide, the Suppressor oligonucleotide binding to the Template molecule was irreversible and both mutant Template molecules and wildtype Template molecules were unable to amplify. Only when both Suppressor oligonucleotide and Auxiliary oligonucleotide were present was there differential amplification of mutant and wildtype Template molecules. In other words, when both Suppressor oligonucleotide and Auxiliary oligonucleotide were present, then preferential amplification of the NA18537 was observed, because its Template molecule was mismatched against the Suppressor oligonucleotide.

To demonstrate that ACE enriches all Template molecules with sequence variations within the Target Subsequence, an ACE system was designed to target the human TP53 gene (FIG. 7). The Suppressor oligonucleotide for the ACE set had the sequence: GGGTCACTGCCATGGAGGAGCCGCAGTCAGATCCTAGCGTCGAGCCCCCTCTGA GTCAGGAAACATTTTCAGACCCGCAACG/3SpC3/(SEQ ID NO: 38). The Auxiliary oligonucleotide for the ACE set had the sequence: CGTTGCGGGTCTGAAAATGTTTCCTGACTCAGAGGGGGCTCGACGCTAGGATCTG ACTGCGGCTCCTC/3SpC3/(SEQ ID NO: 39). This ACE system was tested using qPCR using 15 separate TP53 mutations at different loci spanning across the 50 nt Target Subsequence. This experiment was performed using synthetic gBlock oligonucleotide templates (606 nt long each). For each qPCR reaction, 2500 molecules of each gBlock with the mutation of interest were used as 100% VAF sample. A Taqman probe (/5Cy5/AATGGATCCACTCACAGTTTCCATA/3IAbRQSp/; SEQ ID NO: 40) that bind downstream of the Suppressor oligonucleotide was used to produce a specific fluorescence signal for the amplicons generated. The observed Ct values of 7.5 ng NA18537 without mutant Template spike-in were plotted as a line and have values of about 40. The Ct values of the spike-in samples were all roughly 9 to 13 cycles lower, at between 27 and 31 (FIG. 7). Based on the design of the ACE mechanism, all mutations were selectively enriched by the same ACE Suppressor oligonucleotide and Auxiliary oligonucleotide regardless of the mutation's position on the Suppressor oligonucleotide.

To demonstrate that the length of the Target Subsequence can be extended through rational design of the Suppressor oligonucleotide and the Auxiliary oligonucleotide, three separate ACE sets targeting the same human SNP (rs1443486) with different Target Subsequence lengths of 64 nt (51+1+12), 81 nt, and 126 nt were constructed (FIG. 8). The Suppressor oligonucleotide for the 126 nt ACE set had the sequence: gccactagcaccatttacagccagagcctctgatcgggagatggtctctcttgggggcgctttcctgcagggaaacagcatcgattgtt ttctttaaaagatcccctactccTttttggctaactGAACCCTGAC/3SpC3/(SEQ ID NO: 41). The Auxiliary oligonucleotide for the 126 nt ACE set had the sequence: GTCAGGGTTCagttagccaaaaAggagtaggggatcttttaaagaaaacaatcgatgctgtttccctgcaggaaagcgcccc caagagagaccatctcccgaagcagaggctctggctgta/3SpC3/(SEQ ID NO: 42). The Suppressor oligonucleotide for the 81 nt ACE set had the sequence: tctctcttgggggcgctttcctgcagggaaacagcatcgattgttttctttaaaagatcccctactccTttttggctaactGAACCCT GAC/3SpC3/(SEQ ID NO: 43). The Auxiliary oligonucleotide for the 81 nt ACE set had the sequence: GTCAGGGTTCagttagccaaaaAggagtaggggatcttttaaagaaaacaatcgatgctgtttccctgcaggaaagc/3SpC3/(SEQ ID NO: 44). The Suppressor oligonucleotide for the 64 nt ACE set had the sequence: ttcctgcagggaaacagcatcgattgttttctttaaaagatcccctactccTttttggctaactGAACCCTGACTT/3 SpC3/(SEQ ID NO: 35). The Auxiliary oligonucleotide for the 64 nt ACE set had the sequence: GTCAGGGTTC agttagccaaaaAggagtaggggatcttttaaagaaaacaatcgatgct/3 SpC3/(SEQ ID NO: 36).

The SNP position was designed to be consistently the 13th nucleotide from the end of the Template-binding region. All three ACE systems showed significant Ct value differences between the NA18537 variant Template and the NA18562 wildtype Template. Significant delay was observed for the longest Suppressor oligonucleotide, suggesting either that that longer length necessitates a longer anneal cycle time to allow strand displacement, or that the longer length causes the Auxiliary oligonucleotide purity to drop due to the lower purities of longer chemically synthesized oligonucleotides, rendering displacement less efficient.

### Example 3—Next Generation Sequencing (NGS) Library Preparation Protocols

The data for the NGS experiments summarized in FIG. 10 were collected using an Illumina MiSeq instrument and a MiSeq v3 single-end 150 cycle kit. Each library used 25 ng input DNA in 50 μL reaction mixture. The library preparation process is briefly summarized below:

1. Perform 33 cycles (95° C. for 30 seconds, 60° C. for 6 minutes) of ACE-PCR using PowerUP mastermix (Thermo Fisher), using the Forward Primer oligonucleotide, Reverse Primer oligonucleotide, Suppressor oligonucleotide, and Auxiliary oligonucleotide concentrations listed in FIG. 10.

2. Perform DNA purification using 1.7×SPRI beads.

3. Perform 2 cycles of adapter PCR (95° C. for 10 seconds, 60° C. for 6 minutes) using iTaq mastermix (Bio-Rad), using 15 nM primer per plex.

4. Perform DNA purification using 1.4×SPRI beads.

5a. For libraries without Suppressor oligonucleotide, perform 8 cycles index PCR (95° C. for 10 seconds, 60° C. for 30 seconds) using iTaq mastermix (Bio-Rad), using 500 nM index primers.

5b. For libraries with Suppressor oligonucleotide, perform 10 cycles for index PCR (95° C. for 10 seconds, 60° C. for 30 seconds) iTaq mastermix (Bio-Rad), using 500 nM index primers.

6. Perform DNA purification using 1.1×SPRI beads.

### Example 4—NGS Bioinformatic Analysis Methods

The method for analyzing NGS reads from NGS FASTQ files is summarized below:

1. Trim adapters sequences from each read.

2. Count the number of insert reads that perfectly match the wildtype amplicon (WT Reads) or variant amplicon (Var Reads). Any degenerate nucleotides in the reads, such as N, are considered mismatched and do not contribute to WT Reads or Var Reads. The fraction of all NGS reads in the library that can be counted as a WT Read or Var Read for any locus is here defined as the On-Target Rate.

3. The variant read frequency (VRF) of an amplicon is calculated as:

VRF=(Var Reads)/(Var Reads+WT Reads)

### Example 5—ACE in Next Generation Sequencing (NGS) Library Preparation

ACE can be used for variant enrichment during the library preparation process of a high-throughput sequencing procedure, such as, for example, in a sequencing-by-synthesis (NGS) method. Alternatively, the high-throughput sequencing may be performed via electrical current measurements in conjunction with a nanopore.

An 18-plex ACE panel (Table 1), targeting 18 different SNP loci in which NA18537 and NA18562 were homozygous for different alleles, was constructed. This 18-plex ACE panel was designed to suppress the homozygous SNP alleles of the NA18562 sample. All 18 Suppressor oligonucleotides were designed to be perfectly matched against the NA18562 alleles. The 18-plex ACE was tested on a sample of 1% NA18537/99% NA18562; each library used 25 ng of this mixture as input. An Illumina MiSeq was used for performing NGS. Without the ACE Suppressor oligonucleotide and Auxiliary oligonucleotide, the number of NGS reads mapping to the NA18562 allele was roughly 100-fold higher than the number of NGS reads mapping to the NA18537 allele at every locus, as expected (FIG. 10). In the libraries with the ACE Suppressor oligonucleotide and Auxiliary oligonucleotide present, the relative fraction of reads mapping to the NA18537 variant allele was significantly increased for all loci. Overall, the fraction of NGS reads mapped to the NA18537 loci was increased from 1.22% to up to 33.8%, a weighted average enrichment of more than 24-fold. Based on these single-plex qPCR ACE results, the ACE fold-enrichment can be significantly further improved through the optimization of sequences, concentrations, reaction times, and other experimental protocol minutiae.

TABLE 1
Oligonucleotides for the 18-plex ACE panel.

| | | | SEQ ID |
|---|---|---|---|
| Locus | Oligo Type | Oligo Sequence | NO: |
| rs11247921 | Auxiliary | TAACACATTGATGcagaaaaacaGcatacca | 1 |
| | | tgagaggcagagtgtggaagtcagagaaacc | |
| | | c/3SpC3/ | |
| rs11247921 | Suppressor | Tctcctcctccaggagggtttctctgacttc | 2 |
| | | cacactctgcctctcatggtatgCtgttttt | |
| | | ctgCATCAATGTGTTAAC/3SpC3/ | |
| rs2246745 | Auxiliary | CGTCAAGGcaaacatgccAtctccttctcct | 3 |
| | | gattattttacatggaatctcacctggat/ | |
| | | 3SpC3/ | |
| rs2246745 | Suppressor | Gggaaggagtctttcattatccaggtgagat | 4 |
| | | tccatgtaaaataatcaggagaaggagaTgg | |
| | | catgtttgCCTTGACGC/3SpC3/ | |
| rs16754 | Auxiliary | CGTCTGAGATAGTAaggatgtgcgGcgtgtg | 5 |
| | | cctggagtagccccgactcttgtacggtcgg | |
| | | ca/3SpC3/ | |
| rs16754 | Suppressor | Ttctcactggtctcagatgccgaccgtacaa | 6 |
| | | gagtcggggctactccaggcacacgCcgcac | |
| | | atcctTACTATCTCAGACGG/3SpC3/ | |
| rs2301720 | Auxiliary | TACTGCAGGgctcctttgcGcccaactcaca | 7 |
| | | gagaagcggctacggggcgggcgccgg/ | |
| | | 3SpC3/ | |
| rs2301720 | Suppressor | Cgaggcgaaggcgccggcgcccgccccgtag | 8 |
| | | ccgcttctctgtgagttgggCgcaaaggagc | |
| | | CCTGCAGTAATC/3SpC3/ | |
| rs1123828 | Auxiliary | AGTGGCACAtcaaacacccGtgctcaccctt | 9 |
| | | ccccttcctcgtctacatg/3SpC3/ | |
| rs1123828 | Suppressor | Gtactaacccatgggccatgtagacgaggaa | 10 |
| | | ggggaagggtgagcaCgggggGG | |
| | | CACTCA/3SpC3/ | |
| rs3813787 | Auxiliary | ATGATGGTGAAGTagttcaagctcGacccca | 11 |
| | | gccaagtccgattccgaagccctgagg/ | |
| | | 3SpC3/ | |
| rs3813787 | Suppressor | Ggattcggtctggccctcagggcttcggaat | 12 |
| | | cggacttggctggggtCgagcttgaactACT | |
| | | TCACCATCATTAC/3SpC3/ | |
| rs2170091 | Auxiliary | TAGTGCGAGTtggaatgtGtctgaagctatc | 13 |
| | | tatgaagagcaagatgggaaggagattat/ | |
| | | 3SpC3/ | |
| rs2170091 | Suppressor | Acatgagagggctctaaataatctccttccc | 14 |
| | | atcttgctcttcatagatagcttcagaCaca | |
| | | ttccaACTCGCACTACT/3SpC3/ | |
| rs7104025 | Auxiliary | CGACAGGACtcatctccttCttaactcatga | 15 |
| | | gcctaaagcatctgattctaggctcatct/ | |
| | | 3SpC3/ | |
| rs7104025 | Suppressor | Agtgttcagactgggaagatgagcctagaat | 16 |
| | | cagatgctttaggctcatgagttaaGaagga | |
| | | gatgaGTCCTGTCG/3SpC3/ | |
| rs28932178 | Auxiliary | GGACTGTCAGttaacctgagAcgtctcggtt | 17 |
| | | ccaggetctgcactcttagtacaaccca/ | |
| | | 3SpC3/ | |
| rs28932178 | Suppressor | Ctcacatacagaccacttaatgggttgtact | 18 |
| | | aagagtgcagagcctggaaccgagacgTctc | |
| | | aggttaaCTGACAGTCCTC/3SpC3/ | |
| rs12681931 | Auxiliary | GCCTCTATCTGAaaactcagaccGatttggc | 19 |
| | | catagattattagctctgagaaacagtgtgt | |
| | | ctga/3SpC3/ | |
| rs12681931 | Suppressor | Cactacacacacactctctcagacacactgt | 20 |
| | | ttctcagagctaataatctatggccaaatCg | |
| | | gtctgagtttTCAGATAGAGGC/3SpC3/ | |
| rs1375977 | Auxiliary | GCCTAGCGtctttgtgaaCgtataaagctgg | 21 |
| | | gtgcttttaggagcacccaagtcacctcttg | |
| | | aat/3SpC3/ | |
| rs1375977 | Suppressor | Ccaagcagcaaagcattcaagaggtgacttg | 22 |
| | | ggtgctcctaaaagcacccagctttatacGt | |
| | | tcacaaagaCGCTAGGC/3SpC3/ | |
| rs2215492 | Auxiliary | GTACAGTGCActgaccatttAatacacatgg | 23 |
| | | ggtaacctttggggcatcctgccattatgtc | |
| | | t/3SpC3/ | |
| rs2215492 | Suppressor | Ccagaggctgtgcagacataatggcaggatg | 24 |
| | | ccccaaaggttaccccatgtgtatTaaatgg | |
| | | tcag TGCACTGTACG/3SpC3/ | |
| rs6937778 | Auxiliary | ACAAGCATGTAATcttgctttccTacaccac | 25 |
| | | taccttttcatgtatcctggcttcgtttcca | |
| | | tgttg/3SpC3/ | |
| rs6937778 | Suppressor | Ttaggtcatttataggcctccaacatggaaa | 26 |
| | | cgaagccaggatacatgaaaaggtagtggtg | |
| | | tAggaaagcaagATTACATGCTTGTCT/ | |
| | | 3SpC3/ | |
| rs7032336 | Auxiliary | CCCACTCAAGTAtgaaagcacgGgaacgtga | 27 |
| | | gttcagaagagagagatatcaaagagg/ | |
| | | 3SpC3/ | |
| rs7032336 | Suppressor | Attccaaatgcttaatggatatttcctcttt | 28 |
| | | gatatctctctcttctgaactcacgttcCcg | |
| | | tgctttcaTACTTGAGTGGG/3SpC3/ | |
| rs7893462 | Auxiliary | GAGGAAGCGatagtgagaaTgagcagctgca | 29 |
| | | ggagcactgcgccatggccatttaccaggtg | |
| | | cagtgaac/3SpC3/ | |
| rs7893462 | Suppressor | Aaccgctgggagagttcactgcacctggtaa | 30 |
| | | atggccatggcgcagtgctcctgcagctgct | |
| | | cAttctcactatCGCTTCCTCC/3SpC3/ | |
| rs206781 | Auxiliary | CTTGAACAGGTgtccaaagccAgaagggcct | 31 |
| | | aaagcagcactgccacccccactgccacttg | |
| | | ctt/3SpC3/ | |
| rs206781 | Suppressor | Caacctaagaagtccaagaaagcaagtggca | 32 |
| | | gtgggggtggcagtgctgctttaggcccttc | |
| | | TggctttggacACCTGTTCAAG/3SpC3/ | |
| rs10104396 | Auxiliary | ATGTCCGAAGtagctattttAtcacatagtc | 33 |
| | | attcttctaatacccctctgctca/ | |
| | | 3SpC3/ | |
| rs10104396 | Suppressor | Agcatagggaagaagaattagtgagcagagg | 34 |
| | | ggtattagaagaatgactatgtgaTaaaata | |
| | | gctaCTTCGGACATCA/3SpC3/ | |

All of the methods disclosed and claimed herein can be made and executed without undue experimentation in light of the present disclosure. While the compositions and methods of this invention have been described in terms of preferred embodiments, it will be apparent to those of skill in the art that variations may be applied to the methods and in the steps or in the sequence of steps of the method described herein without departing from the concept, spirit and scope of the invention. More specifically, it will be apparent that certain agents which are both chemically and physiologically related may be substituted for the agents described herein while the same or similar results would be achieved. All such similar substitutes and modifications apparent to those skilled in the art are deemed to be within the spirit, scope and concept of the invention as defined by the appended claims.

What is claimed is:

1. A composition comprising:
(a) an Auxiliary oligonucleotide,
(b) a Suppressor oligonucleotide, wherein the Suppressor oligonucleotide comprises a Protected Subsequence that is at least 20 nucleotides long and that is reverse complementary to a subsequence of the Auxiliary oligonucleotide, wherein the Suppressor oligonucleotide comprises an Unprotected Subsequence that is at least 7 nucleotides long and that is not reverse complementary to the Auxiliary oligonucleotide,
(c) a Forward Primer oligonucleotide, wherein the Forward Primer oligonucleotide comprises an at least 6 nucleotide long subsequence that is identical to a subsequence of the Suppressor oligonucleotide, and
(d) a template-dependent polymerase.

2. The composition of claim 1, wherein the Auxiliary oligonucleotide comprises DNA.

3. The composition of claim 1 or 2, wherein the Auxiliary oligonucleotide consists of DNA.

4. The composition of any one of claims 1-3, wherein the Suppressor oligonucleotide comprises DNA.

5. The composition of any one of claims 1-4, wherein the Suppressor oligonucleotide consists of DNA.

6. The composition of claim 1, wherein the Auxiliary oligonucleotide comprises non-natural oligonucleotides.

7. The composition of claim 1 or 6, wherein the Suppressor oligonucleotide comprises non-natural oligonucleotides.

8. The composition of any one of claims 1-7, wherein the Forward Primer oligonucleotide comprises DNA.

9. The composition of any one of claims 1-8, wherein the Forward Primer oligonucleotide consists of DNA.

10. The composition of any one of claims 1-9, wherein the template-dependent polymerase is a DNA polymerase.

11. The composition of any one of claims 1-9, wherein the template-dependent polymerase is a reverse transcriptase.

12. The composition of any one of claims 1-7, wherein the Forward Primer oligonucleotide comprises RNA.

13. The composition of any one of claims 1-7 and 12, wherein the Forward Primer oligonucleotide consists of RNA.

14. The composition of claim 12 or 13, wherein the template-dependent polymerase is an RNA polymerase.

15. The composition of any one of claims 1-14, wherein the Suppressor oligonucleotide has a length between 30 and 500 nucleotides.

16. The composition of any one of claims 1-15, wherein the Unprotected Subsequence of the Suppressor oligonucleotide is not reverse complementary to any portion of the Auxiliary oligonucleotide.

17. The composition of any one of claims 1-16, wherein the Auxiliary oligonucleotide has a length between 30 and 500 nucleotides.

18. The composition of any one of claims 1-17, wherein the Forward Primer oligonucleotide has a length between 6 and 70 nucleotides.

19. The composition of any one of claims 1-18, wherein the Suppressor oligonucleotide has a 3′ chemical modification or DNA sequence that prevents template-dependent polymerase extension.

20. The composition of any one of claims 1-19, wherein the Auxiliary oligonucleotide has a 3′ chemical modification or DNA sequence that prevents template-dependent polymerase extension.

21. The composition of claim 19 or 20, wherein the modification comprises a dideoxynucleotide, inverted DNA nucleotides, phosphorothioate-substituted backbone, and alkane or polyethylene glycol (PEG) spacers.

22. The composition of claim 19 or 20, wherein the DNA sequence at the 3′ end forms at least one hairpin structure.

23. The composition of any one of claims 1-22, wherein the composition comprises a plurality of Suppressor oligonucleotide species, a plurality of Auxiliary oligonucleotide species, and a plurality of Forward Primer oligonucleotide species.

24. The composition of claim 23, wherein each Suppressor oligonucleotide species comprises a Protected Subsequence that is at least 20 nucleotides long and that is reverse complementary to a subsequence of at least one corresponding Auxiliary oligonucleotide species.

25. The composition of claim 23 or 24, wherein each Forward Primer oligonucleotide species comprises an at least 6 nucleotide long subsequence that is identical to a subsequence of at least one corresponding Suppressor oligonucleotide species.

26. The composition of any one of claims 23-25, wherein the plurality of Forward Primer oligonucleotide species each comprises a Universal Forward Adapter subsequence at or near the 5′ end.

27. The composition of any one of claims 1-26, further comprising a nucleic acid Template molecule, wherein the Template molecule comprises a subsequence that is over 90% homologous to the reverse complement of the 3′ subsequence of the Forward Primer oligonucleotide.

28. The composition of any one of claims 1-27, further comprising a Reverse Primer oligonucleotide, wherein the Template molecule comprises a subsequence that is over 90% homologous to a 3′ subsequence of the Reverse Primer oligonucleotide.

29. The composition of claim 28, wherein the Reverse Primer oligonucleotide has a length between 10 and 70 nucleotides.

30. The composition of claim 28-29, wherein the Reverse Primer oligonucleotide comprises a Universal Reverse Adapter subsequence at or near the 5′ end.

31. The composition of any one of claims 27-30, wherein the Template molecule is a biological DNA or RNA molecule.

32. The composition of any one of claims 27-31, wherein the Template molecule is obtained from a sample of cells.

33. The composition of any one of claims 27-31, wherein the Template molecule is obtained from a biofluid.

34. The composition of claim 33, wherein the biofluid is blood, urine, saliva, cerebrospinal fluid, interstitial fluid, or synovial fluid.

35. The composition of any one of claims 27-31, wherein the Template molecule is obtained from a tissue.

36. The composition of claim 35, wherein the tissue is a biopsy tissue or a surgically resected tissue.

37. The composition of any one of claims 27-36, wherein the Template molecule is a complementary DNA molecule generated through the reverse transcription of an RNA molecule.

38. The composition of claim 37, wherein the RNA molecule is obtained from a biological RNA sample derived from a human, animal, plant, or environmental specimen.

39. The composition of any one of claims 27-36, wherein the Template molecule is an amplicon DNA molecule generated through a DNA polymerase acting on a single-stranded DNA template.

40. The composition of claim 39, wherein the Template molecule is an amplicon DNA molecule generated from multiple displacement amplification of a single cell DNA.

41. The composition of any one of claims 27-36, wherein the Template molecule is a physically, chemically, or enzymatically generated product of a biological DNA molecule.

42. The composition of claim 41, wherein the Template molecule is the product of a fragmentation process.

43. The composition of claim 42, wherein the fragmentation process is ultrasonication or enzymatic fragmentation.

44. The composition of claim 41, wherein the Template molecule is the product of a bisulfite conversion reaction, an APOBEC reaction, a TAPS reaction, or other chemical or enzymatic reaction in which cytosine nucleotides are selectively converted to uracil nucleotides based on its methylation status.

45. The composition of any one of claims 27-44, wherein the Template molecule comprises a Target Subsequence positioned between a Forward Primer-binding Subsequence and a Reverse Primer-homologous Subsequence.

46. The composition of claim 45, wherein the Target Subsequence is at least 70% identical to the reverse complement of the portion of Suppressor oligonucleotide Protected Subsequence that does not include the Initiation Subsequence.

47. The composition of claim 45 or 46, wherein the Suppressor oligonucleotide has an Initiation Subsequence at or near the 3′ of the Suppressor oligonucleotide.

48. The composition of claim 47, wherein the Initiation Subsequence has a length between 4 and 25 nucleotides.

49. The composition of claim 47 or 48, wherein the Initiation Subsequence is less than 30% identical to the reverse complement of the Template molecule subsequence that is immediately to the 3′ of the Target Subsequence.

50. The composition of any one of claims 47-49, wherein the Auxiliary oligonucleotide has an Initiation Complement Subsequence at or near the 5′ end of the Auxiliary oligonucleotide.

51. The composition of claim 50, wherein the Initiation Complement Subsequence has a length between 4 and 25 nucleotides.

52. The composition of claim 50 or 51, wherein the Initiation Complement Subsequence is at least 90% identical to the reverse complement of the Initiation Subsequence of the Suppressor oligonucleotide.

53. The composition of any one of claims 27-52, wherein the Auxiliary oligonucleotide does not have a subsequence that is more than 30% identical to the reverse complement Forward Primer oligonucleotide.

54. The composition of any one of claims 1-53, further comprising a fluorophore-functionalized DNA probe.

55. The composition of claim 54, wherein the fluorophore-functionalized DNA probe is a Taqman probe or a molecular beacon.

56. The composition of any one of claims 1-53, further comprising a DNA intercalating dye.

57. The composition of claim 56, wherein the DNA intercalating dye is SybrGreen, EvaGreen, or Syto.

58. The composition of any one of claims 1-57, wherein the stoichiometric ratio of the Auxiliary oligonucleotide to the Suppressor oligonucleotide is between 0.8 and 100.

59. The composition of any one of claims 1-58, wherein the Forward Primer oligonucleotide and the Template molecule have a standard free energy of hybridization (ΔG°₁) between −7 kcal/mol and −20 kcal/mol at a temperature of 60° C. and a salinity of 0.2 M sodium.

60. The composition of any one of claims 1-59, wherein the Suppressor oligonucleotide and the Template molecule have a standard free energy of hybridization (ΔG°₂) between −16 kcal/mol and −200 kcal/mol at a temperature of 60° C. and a salinity of 0.2 M sodium.

61. The composition of any one of claims 1-60, wherein the Suppressor oligonucleotide and the Auxiliary oligonucleotide have a standard free energy of hybridization (ΔG°₃) between −15 kcal/mol and −200 kcal/mol at a temperature of 60° C. and a salinity of 0.2 M sodium.

62. The composition of any one of claims 60 and 61, wherein the value of (ΔG°₂−ΔG°₃) is between −5 kcal/mol and +5 kcal/mol.

63. The composition of any one of claims 1-62, further comprising reagents and buffers needed for polymerase function.

64. A method for selectively amplifying a DNA sequence variant using polymerase chain reaction, the method comprising:
(a) mixing a Sample possibly comprising a variant DNA Template molecule and possibly comprising a wildtype DNA Template molecule with:
(i) an Auxiliary oligonucleotide,
(ii) a Suppressor oligonucleotide, wherein the Suppressor oligonucleotide comprises a Protected Subsequence that is at least 20 nucleotides long and that is reverse complementary to a subsequence of the Auxiliary oligonucleotide, wherein the Suppressor oligonucleotide comprises an Unprotected Subsequence that is at least 7 nucleotides long and that is not reverse complementary to the Auxiliary oligonucleotide, wherein the Protected Subsequence comprises a Target-binding Subsequence and an Initiation Subsequence, and wherein the Target-binding Subsequence is at least 70% identical to the reverse complement of the wildtype DNA Template molecule,
(iii) a Forward Primer oligonucleotide, wherein the Forward Primer oligonucleotide comprises an at least 6 nucleotide long subsequence that is identical to at least a portion of the Unprotected Subsequence of the Suppressor oligonucleotide, and wherein the reverse complement of 3′ subsequence of the Forward Primer oligonucleotide is at least 90% identical to the wildtype DNA Template,
(iv) a Reverse Primer oligonucleotide, wherein the 3′ subsequence of the Reverse Primer oligonucleotide is at least 90% identical to a subsequence of the wildtype DNA Template, and
(v) a template-dependent DNA polymerase, dNTPs, and buffer reagents needed for DNA polymerase function, and
(b) subjecting the mixture to at least 7 rounds of thermal cycling.

65. The method of claim 64, wherein each round of thermal cycling comprises holding the mixture at a denaturing temperature of between 80° C. and 105° C. for between 1 second and 1 hour and then holding the mixture at an annealing temperature of between 50° C. and 75° C. for between 1 second and 2 hours.

66. The method of claim 64 or 65, wherein a plurality of Forward Primer oligonucleotides, Reverse Primer oligonucleotides, Suppressor oligonucleotides, and Auxiliary oligonucleotides are mixed with the Sample, wherein each set of Forward Primer oligonucleotides, Reverse Primer oligonucleotides, Suppressor oligonucleotides, and Auxiliary oligonucleotides corresponds to different variant Template molecule and wildtype Template molecule sequences.

67. The method of any one of claims 64-66, wherein all Forward Primer oligonucleotides comprise a Universal Forward Adapter subsequence at or near the 5′ end, and wherein all Reverse Primer oligonucleotides comprise a Universal Reverse Adapter subsequence at or near the 5′ end.

68. The method of any one of claims 64-67, wherein each Suppressor oligonucleotide species comprises a Protected Subsequence that is at least 20 nucleotides long and that is reverse complementary to a subsequence of at least one corresponding Auxiliary oligonucleotide species.

69. The method of any one of claims 64-68, wherein each Forward Primer oligonucleotide species comprises an at least 6 nucleotide long subsequence that is identical to a subsequence of at least one corresponding Suppressor oligonucleotide species.

70. The method of any one of claims 64-69, wherein the Forward Primer oligonucleotide and the variant Template molecule have a standard free energy of hybridization (ΔG°₁) between −7 kcal/mol and −20 kcal/mol at a temperature of 60° C. and a salinity of 0.2 M sodium.

71. The method of any one of claims 64-70, wherein the Suppressor oligonucleotide and the wildtype Template molecule have a standard free energy of hybridization (ΔG°₂) between −16 kcal/mol and −200 kcal/mol at a temperature of 60° C. and a salinity of 0.2 M sodium.

72. The method of any one of claims 64-71, wherein the Suppressor oligonucleotide and the Auxiliary oligonucleotide have a standard free energy of hybridization (ΔG°₃) between −15 kcal/mol and −200 kcal/mol at a temperature of 60° C. and a salinity of 0.2 M sodium.

73. The method of any one of claims 71 and 72, wherein the value of (ΔG°₂−ΔG°₃) is between −5 kcal/mol and +5 kcal/mol.

74. The method of any one of claims 64-73, wherein the Reverse Primer oligonucleotide and the variant Template molecule have a standard free energy of hybridization (ΔG°₄) between −7 kcal/mol and −20 kcal/mol at a temperature of 60° C. and a salinity of 0.2 M sodium.

75. The method of any one of claims 64-74, wherein the concentration of each Forward Primer oligonucleotide in the mixture is between 100 pM and 5 μM.

76. The method of any one of claims 64-75, wherein the concentration of each Reverse Primer oligonucleotide in the mixture is between 100 pM and 5 μM.

77. The method of any one of claims 64-76, wherein the concentration of each Suppressor oligonucleotide in the mixture is between 100 pM and 5 μM.

78. The method of any one of claims 64-77, wherein the concentration of each Auxiliary oligonucleotide is between 100 pM and 5 μM.

79. The method of any one of claims 64-78, wherein the stoichiometric ratio of each Forward Primer oligonucleotide to its corresponding Suppressor oligonucleotide is between 0.8 and 100.

80. The method of any one of claims 64-79, wherein the stoichiometric ratio of each Auxiliary oligonucleotide to its corresponding Suppressor oligonucleotide is between 0.8 and 100.

81. The method of any one of claims 64-80, wherein the Auxiliary oligonucleotide comprises DNA.

82. The method of any one of claims 64-81, wherein the Auxiliary oligonucleotide consists of DNA.

83. The method of any one of claims 64-82, wherein the Suppressor oligonucleotide comprises DNA.

84. The method of any one of claims 64-83, wherein the Suppressor oligonucleotide consists of DNA.

85. The method of any one of claims 64-84, wherein the Suppressor oligonucleotide has a length between 30 and 500 nucleotides.

86. The method of any one of claims 64-85, wherein the Unprotected Subsequence of the Suppressor oligonucleotide is not reverse complementary to any portion of the Auxiliary oligonucleotide.

87. The method of any one of claims 64-86, wherein the Auxiliary oligonucleotide has a length between 30 and 500 nucleotides.

88. The method of any one of claims 64-87, wherein the Forward Primer oligonucleotide has a length between 6 and 70 nucleotides.

89. The method of any one of claims 64-88, wherein the Reverse Primer oligonucleotide has a length between 10 and 70 nucleotides.

90. The method of any one of claims 64-89, wherein the Suppressor oligonucleotide has a 3′ chemical modification or DNA sequence that prevents DNA polymerase extension.

91. The method of any one of claims 64-90, wherein the Auxiliary oligonucleotide has a 3′ chemical modification or DNA sequence that prevents DNA polymerase extension.

92. The method of claim 90 or 91, wherein the modification comprises dideoxynucleotides, inverted DNA nucleotides, phosphonothioate-substituted backbone, and alkane or polyethylene glycol (PEG) spacers.

93. The method of claim 90 or 91, wherein the DNA sequence at the 3′ end forms at least one hairpin structure.

94. The method of any one of claims 64-93, wherein the Suppressor oligonucleotide has an Initiation Subsequence at or near the 3′ of the Suppressor oligonucleotide.

95. The method of any one of claims 64-94, wherein the Initiation Subsequence has a length between 4 and 25 nucleotides.

96. The method of any one of claims 64-95, wherein the Initiation Subsequence is less than 30% identical to the reverse complement of the variant Template molecule subsequence that is immediately to the 3′ of the Target Subsequence.

97. The method of any one of claims 64-96, wherein the Auxiliary oligonucleotide has an Initiation Complement Subsequence at or near the 5′ end of the Auxiliary oligonucleotide.

98. The method of claim 97, wherein the Initiation Complement Subsequence has a length between 4 and 25 nucleotides.

99. The method of claim 97 or 98, wherein the Initiation Complement Subsequence is at least 90% identical to the reverse complement of the Initiation Subsequence of the Suppressor oligonucleotide.

100. The method of any one of claims 64-99, wherein the Auxiliary oligonucleotide does not have a subsequence that is more than 30% identical to the reverse complement of the Forward Primer oligonucleotide.

101. The method of any one of claims 64-100, wherein the mixture further comprises a fluorophore-functionalized DNA probe.

102. The method of claim 101, wherein the fluorophore-functionalized DNA probe is a Taqman probe or a molecular beacon.

103. The method of any one of claims 64-100, wherein the mixture further comprises a DNA intercalating dye.

104. The method of claim 103, wherein the DNA intercalating dye is SybrGreen, EvaGreen, or Syto.

105. A method for selectively detecting and quantifying DNA sequence variants using quantitative PCR (qPCR), the method comprising:
(a) performing selective PCR amplification of variant DNA templates over wildtype DNA templates in a first aliquot of a Sample according to the method of any one of claims 64-104;
(b) performing time-based measurements of solution fluorescence;
(c) calculating a cycle threshold (Ct) value based on the cycle in which the solution fluorescence exceeds a threshold; and
(d) making a determination of the presence/absence or quantity of the variant DNA Template in the Sample based on the Ct value.

106. The method of claim 105, wherein the qPCR mixture comprises a Taqman probe.

107. The method of claim 105 or 106, further comprising:
(e) performing a second qPCR reaction on a second aliquot of the Sample using the Forward Primer oligonucleotide and the Reverse Primer oligonucleotide, in the absence of Suppressor oligonucleotide;
(f) calculating a cycle threshold (Ct2) of this second reaction; and
(g) making a determination on the relative quantity of variant DNA Template to wildtype DNA Template based on the difference in values between Ct and Ct2.

108. A method for selectively detecting and quantifying DNA sequence variants using high-throughput sequencing, the method comprising:
(a) performing selective PCR amplification of variant DNA Templates over wildtype DNA Templates in a first aliquot of a Sample according to the method of any one of claims 64-104;
(b) appending sequencing adapters to either or both ends of the amplicons; and
(c) performing high-throughput sequencing on the product of step (b); and
(d) determining the mutation VAF of the Sample based on the high-throughput sequencing reads.

109. The method of claim 108, wherein the Forward Primer oligonucleotide comprises a forward sequencing adapter at its 5′ end, and the Reverse Primer oligonucleotide comprises a reverse sequencing adapter at its 5′ end.

110. The method of claim 109, wherein one or both of the sequencing adapters comprise unique molecular identifier (UMI) sequences.

111. The method of claim 108, further comprising appending sequencing adapters and/or sequencing indexes using PCR.

112. The method of claim 111, wherein the sequencing adapters comprise unique molecular identifier (UMI) sequences.

113. The method of claim 108, further comprising ligating sequencing adapters and/or sequencing indexes to the PCR product of step (a) before performing high-throughput sequencing.

114. The method of claim 113, wherein the sequencing adapters appended via ligation comprise unique molecular identifier (UMI) sequences.

115. The method of claim 110, 112, or 114, wherein the UMI sequences comprise a set of pre-designed sequences wherein every pair of UMI sequences exhibit a minimal Hamming distance that is not less than 30% of the length of the UMI.

116. The method of claim 110, 112, 114, or 115, wherein the UMI sequences comprise a set of sequences comprising degenerate nucleotides, selected from N (mixture of A, C, G, and T), B (mixture of C, G, and T), D (mixture of A, G, and T), H (mixture of C, A, and T), V (mixture of A, C, and G), S (mixture of C and G), W (mixture of A and T), R (mixture of A and G), Y (mixture of T and C), K (mixture of G and T), and M (mixture of A and C).

117. The method of any one of claims 108-116, wherein the mutation VAF of the Sample is the fraction of variant Template molecules in all Template molecules.

118. The method of claim 117, wherein the determination of mutation VAF is based on variant reads frequency (VRF) and fold-enrichment (EF).

119. The method of claim 117, wherein the determination of mutation VAF is based on UMI clustering.

120. The method of any one of claims 108-119, wherein the high-throughput sequencing is performed via sequencing-by-synthesis.

121. The method of any one of claims 108-119, wherein the high-throughput sequencing is performed via electrical current measurements in conjunction with a nanopore.
